(19) United States
(12) Patent Application Publication
(10) Pub. No.: US 2009/0149952 A1
(43) Pub. Date: Jun. 11, 2009

(54) Intraocular Lenses and Business Methods

(76) Inventor: John H. Shadduck, Tiburon, CA (US)

Correspondence Address: SHAY GLENN LLP, 2755 CAMPUS DRIVE, SUITE 210, SAN MATEO, CA, 94403, US

(21) Appl. No.: 12/347,816

(22) Filed: Dec. 31, 2008

Related U.S. Application Data
(63) Continuation of application No. 11/507,946, filed on Aug. 21, 2006.
Application No. 11/507,946 is a continuation-in-part of application No. 10/358,038, filed on Feb. 3, 2003.
Application No. 10/358,038 is a continuation-in-part of application No. 10/890,576, filed on Jul. 14, 2004.
Application No. 10/890,576 is a continuation-in-part of application No. 11/284,068, filed on Nov. 21, 2005, now Pat. No. 7,278,739.

(51) Int. Cl. A61F 2/16

(52) U.S. Cl. 623/6.37; 623/6.11; 623/6.13

(57) ABSTRACT

An intraocular accommodating lens comprising a resilient polymer monolith with a peripheral component that has a Young's modulus and an equilibrium memory shape that imparts to the capsular sac's periphery the natural shape of the capsule in an accommodated state. The intraocular lens includes a central deformable optic that provides accommodated and disaccommodated shapes. The peripheral component is deformable to a disequilibrium, stressed shape in responsive to equatorial tensioning—and is capable of applying restorative forces to move the lens toward the accommodated shape from a disequilibrium disaccommodated shape. In one embodiment, the central optic portion includes a displaceable media than can be displaced by very low forces of zonular excursion, wherein the displaceable media can comprise a very low modulus polymer or an index-matched fluid.

### CROSS-REFERENCE TO RELATED APPLICATIONS

This application is a continuation of U.S. application Ser. No. 11/507,946, filed Aug. 21, 2006; which is a continuation-in-part of the following applications: U.S. application Ser. No. 10/890,576, filed Oct. 7, 2004; U.S. application Ser. No. 10/358,038, filed Feb. 3, 2003; and U.S. application Ser. No. 11/284,068, filed Nov. 21, 2005, now U.S. Pat. No. 7,278,739. All of the above applications are incorporated by reference as if fully set forth herein.

### BACKGROUND OF THE INVENTION

1. Field of the Invention

The present invention is directed to intraocular lenses and more specifically to lens implants that include a resilient optic that adapts (i.e., accommodates and disaccommodates) in response to normal physiologic zonular de-tensioning and tensioning forces. The optics are designed for refractive lens exchange procedures and combine with a post-phaco capsular sac to provide a biomimetic complex that mimics the energy-absorbing and energy-releasing characteristics of a young still-accommodating lens capsule.

2. Description of the Related Art

The human lens capsule can be afflicted with several disorders that degrade its functioning in the vision system. The most common lens disorder is a cataract which consists of the opacification of the normally clear, natural crystalline lens matrix in a human eye. The opacification usually results from the aging process but can also be caused by heredity or diabetes. FIG. 1A illustrates a lens capsule comprising a capsular sac with an opacified crystalline lens nucleus. In a typical cataract procedure as performed today, the patient's opaque crystalline lens is replaced with a clear lens implant or IOL. (See FIGS. 1A and 1B). The vast majority of cataract patients must wear prescription eyeglasses following surgery to see properly. Conventional IOLs in use today provide the eye with a fixed focal length, wherein focusing on both close-up objects and distant objects is not possible. Intraocular lens implantation for cataracts is the most commonly performed surgical procedure in elderly patients in the U.S. Nearly three million cataract surgeries are performed each year in the U.S., with an additional 2.5 million surgeries in Europe and Asia.

### Mechanisms of Accommodation

Referring to FIG. 1, the human eye defines an anterior chamber 10 between the cornea 12 and iris 14 and a posterior chamber 20 between the iris and the lens capsule 102. The vitreous chamber 30 lies behind the lens capsule. The lens capsule 102 that contains the crystalline lens matrix LM or nucleus has an equator that is attached to cobweb-like zonular ligaments ZL that extend generally radially outward to the ciliary muscle attachments. The lens capsule 102 has transparent elastic anterior and posterior walls or capsular membranes that contain the crystalline lens matrix LM.

Accommodation occurs when the ciliary muscle CM contracts to thereby release the resting zonular tension on the equatorial region of the lens capsule 102. The release of zonular tension allows the inherent elasticity of the lens capsule to alter it to a more globular or spherical shape, with increased surface curvatures of both the anterior and posterior lenticular surfaces. The lens capsule together with the crystalline lens matrix and its internal pressure provides the lens with a resilient shape that is more spherical in an untensioned state. Ultrasound biomicroscopic (UBM) images also show that the apex of the ciliary muscle moves anteriorly and inward-at the same time that the equatorial edge the lens capsule moves inwardly from the sclera during accommodation.

When the ciliary muscle is relaxed, the muscle in combination with the elasticity of the choroid and posterior zonular fibers moves the ciliary muscle into the disaccommodated configuration, which is posterior and radially outward from the accommodated configuration. The radial outward movement of the ciliary creates zonular tension on the lens capsule to stretch the equatorial region of lens toward the sclera. The disaccommodation mechanism flattens the lens and reduces the lens curvature (both anterior and posterior). Such natural accommodative capability thus involves contraction and relaxation of the ciliary muscles by the brain to alter the shape of the lens to the appropriate refractive parameters for focusing the light rays entering the eye on the retina—to provide both near vision and distant vision.

In conventional cataract surgery as depicted in FIGS. 1B and 1C, the crystalline lens matrix is removed leaving intact only the thin walls of the anterior and posterior capsules—together with zonular ligament connections to the ciliary body and ciliary muscles. The crystalline lens core is removed by phacoemulsification through a curvilinear capsularhexis as illustrated in FIG. 1B, i.e., the removal of an anterior portion of the capsular sac. FIG. 1B then depicts a conventional 3-piece IOL just after implantation in the capsular sac.

FIG. 1C next illustrates the capsular sac and the prior art 3-piece IOL after a healing period of a few days to weeks. It can be seen that the capsular sac effectively shrink-wraps around the IOL due to the capsularhexis, the collapse of the walls of the sac and subsequent fibrosis. As can be easily understood from FIGS. 1B and 1C, cataract surgery as practiced today causes the irretrievable loss of most of the eye's natural structures that provide accommodation. The crystalline lens matrix is completely lost-and the integrity of the capsular sac is reduced by the capsularhexis. The “shrink-wrap” of the capsular sac around the IOL can damage the zonule complex, and thereafter it is believed that the ciliary muscles will atrophy.

### Prior Art Pseudo-Accommodative Lens Devices

At least one commercially available IOL, and others in clinical trials, are claimed to “accommodate” even though the capsular sac shrink-wraps around the IOL as shown in FIG. 1 C. If any such prior art lens provides variable focusing power, it is better described as pseudo-accommodation since all the eye's natural accommodation mechanisms of changing the shape of the lens capsule are not functioning. Perhaps the most widely known of the pseudo-accommodative IOLs is a design patented by Cumming which is described in patent disclosures as having hinged haptics that are claimed to flex even after the capsular sac is shrink-wrapped around the haptics. Cumming's patents (e.g., U.S. Pat. Nos. 5,496,366; 5,674,282; 6,197,059; 6,322,589; 6,342,073; 6,387,126) describe the hinged haptics as allowing the lens element to be translated forward and backward in response to ciliary muscle contraction and relaxation within the shrink-wrapped capsule. It is accepted that the movement of such lens is entirely pseudoaccommodative and depends on vitreous displacement that pushes the entire IOL slightly anteriorly with little hinge flex (see: http//www.candcvision.com/ASCRSCCTalks/Slade/Slade.htm). A similar IOL that is implanted in a shrink-wrapped capsule and is sold in Europe by HumanOptics, Spardorfer Strasse 150, 90154 Erlangen, Germany. The HumanOptics lens is the Akkommodative ICU which is not available in the U.S., due to lack of FDA approval. In sum, any prior art IOLs that are implanted in an enucleated, shrink-wrapped lens capsule probably are not flexed by ciliary muscle relaxation, and exhibit only a pseudo-accommodative response due to vitreous displacement.

Since surgeons began using IOLs widely in the 1970's, IOL design and surgical techniques for IOL implantation have undergone a continuous evolution. While less invasive techniques for IOL implantation and new IOL materials technologies have evolved rapidly in the several years, there has been no real development of technologies for combining the capsular sac with biocompatible materials to provide a biomimetic capsular complex. What has stalled all innovations in designing a truly resilient (variable-focus) post-phaco lens capsule has been is the lack of sophisticated materials.

What has been needed are materials and intraocular devices that be introduced into an enucleated lens capsule through a 1 mm. to 2.5 mm. injector, wherein the deployed device and material provide the exact strain-absorbing properties and strain-releasing properties needed to cooperate with natural zonular tensioning forces. Such an intraocular device will allow for the design of dynamic IOLs that can replicate natural accommodation.

### SUMMARY OF THE INVENTION

This invention relates to in-the-capsule implants having a deformable lens that includes interior displaceable media that can be displaced by very limited zonular tensioning, for example less than about 3 grams of radial applied forces about the lens capsule. Finite element analysis (FEA) was utilized to evaluate deformable lens designs. FEA studies determined that the limited forces applied by ciliary muscle relaxation and contraction to a lens capsule were not capable of optimally deforming a uniform modulus polymeric lens that was implanted in a lens capsule—in particular, due to the nature of deformations the occur in a resilient axisymmetric body such as a lens. In an axisymmetric lens body, radial outward tensioning forces result in effectively zero deformation forces at central region of the lens to flatten the lens, which is the region that requires significant deformation to move the lens from a first higher power to a second lower power. FEA studies further determined that high amplitudes of accommodation in response to very limited natural physiologic forces would be possible if (i) a peripheral portion of the lens implant cooperated with the lens capsule to provide the needed restorative forces in response to zonular tensioning and de-tensioning, and (ii) a central optic portion of the lens implant carried a highly displaceable media such as a very low modulus polymer or optionally a flowable media such as an index-matched fluid. FEA studies further determined that deformable lens could be provided with greater accommodative amplitude by deformation and flattening of a peripheral portion of the lens rather than attempting to deform and flatten the central portion of the lens—due the problem of axisymmetry described above.

In one embodiment of the invention, an in-the-capsule lens body is implanted using conventional techniques to create a biomimetic lens capsule complex. The implant is designed to provide the implant/capsular sac complex with a shape and resiliency that mimics the elasticity of a young, still-accommodative lens capsule. The peripheral portion of the implant provides the exact strain-absorbing properties and strain-releasing properties of a natural lens to cooperate with the lens capsule. The implant includes an interior displaceable media that comprises a very low modulus polymer and alternatively can include an interior chamber with a flowable media therein. In another embodiment, the intraocular lens includes a shape memory polymer (SMP) in a peripheral portion of the implant body that can be maintained in a temporary disaccommodated shape for implantation in a lens capsule and for bonding to the disaccommodated lens capsule. Thereafter, a stimulus can return the shape memory polymer to its memory shape corresponds to an accommodated lens shape.

In another aspect of the invention, a business method includes (i) fabricating an in-the-capsule lens that carries an interior displaceable media that is displaceable for deforming the lens surface between first and second powers, (ii) configuring the deformable lens surface for high amplitude axial deformation by the displaceable media about a periphery of a central optic portion and for low amplitude axial deformation about optical axis of the lens, and (iii) collaboratively or independently, marketing the intraocular lens. In this business method, the fabricating step includes fabricating the displaceable media from a polymer having a modulus of less than 1000 KPa, less than 500 KPa and less than 100 KPa. Alternatively, the business method fabricate the lens with a displaceable media that comprises an index-matched fluid in at least one interior chamber in the lens.

In another aspect, a business method of the invention includes (i) fabricating an in-the-capsule lens having a monolithic form with an exterior surface that continuously engages the interior of a capsular sac except the capsularhexis, (ii) configuring the lens with a central optic portion that is deformable between a first power and a second power in response to physiologic accommodating and disaccommodating forces applied to the lens capsule wherein the anterior surface of the optic portion is recessed within the monolithic form, and (iii) collaboratively or independently, marketing the intraocular lens.

In another aspect of the invention, a business method includes (i) fabricating an intraocular lens configured for implantation in a capsular sac, wherein the lens has a monolithic form with an exterior surface that continuously engages the interior of a capsular sac except the capsularhexis, and wherein the form defines at least one interior on-axis, rotationally symmetric interface between at least two elastomeric blocks wherein each block has a different Young's modulus, and (ii) collaboratively or independently, marketing the intraocular lens.

In another aspect of the invention, a business method includes (i) fabricating an intraocular lens configured for implantation in a capsular sac, wherein the lens has a monolithic form with an exterior surface that continuously engages the interior of a capsular sac except the capsularhexis, and wherein a peripheral portion of the lens includes an elastomeric block of a shape memory polymer, and (ii) collaboratively or independently, marketing the intraocular lens.

These and other aspects of the present invention will become readily apparent upon further review of the following drawings and specification.

### BRIEF DESCRIPTION OF THE DRAWINGS

In order to better understand the invention and to see how it may be carried out in practice, some preferred embodiments are next described, by way of non-limiting examples only, with reference to the accompanying drawings, in which like reference characters denote corresponding features consistently throughout similar embodiments in the attached drawings.

FIG. 1A is a perspective cut-away view of an eye with an opacified lens capsule.

FIG. 1B is a perspective cut-away view of the eye of FIG. 1A with a curvilinear capsularhexis and the crystalline lens matrix removed by phacoemulsification, together with the implantation of a prior art 3-piece IOL.

FIG. 1C is a perspective cut-away view of the eye of FIG. 1B showing the lens capsule after wound healing wherein the lens capsule shrink-wraps around the prior art IOL.

FIGS. 2A-2B are perspective schematic views of a uniform modulus elastomeric monolith in equilibrium and disequilibrium shapes.

FIGS. 3A-3B are perspective schematic views of an elastomeric monolith with block portions having different Young's moduli in equilibrium and disequilibrium shapes.

FIGS. 4A-4B are perspective schematic views of another elastomeric monolith with block portions having different Young's moduli in equilibrium and disequilibrium shapes.

FIGS. 5A-5B are views of another elastomeric monolith with block portions having different Young's moduli in equilibrium and disequilibrium shapes.

FIGS. 6A-6B are perspective schematic views of a higher modulus elastomer block that functions as a cam-type actuator to deform a lower modulus block.

FIGS. 7A-7B are perspective schematic views of a higher modulus elastomer block that functions as a lever-type actuator to deform a lower modulus block.

FIG. 8 is a perspective schematic view of an elastomeric monolith with an flexible-arm actuator within a low modulus polymer block in a first position.

FIG. 9 is a view of the elastomeric monolith of FIG. 8 with the flexible-arm actuator in a second position deplacing the lower modulus block.

FIG. 10 is a perspective view of an elastomeric monolith with a curvilinear higher modulus block for applying force to a lower modulus block.

FIG. 11 is a perspective view of an elastomeric monolith with a planar higher modulus block for applying force to a lower modulus block.

FIG. 12 is a view of an elastomeric monolith with a plurality of planar higher modulus blocks for applying forces in a plurality of vectors to a lower modulus block.

FIG. 13 is a view of an elastomeric optic monolith similar to that of FIG. 12 with a variable stiffness surface layer.

FIGS. 14A and 14B are views of an assembled and disassembled elastomeric monolith with block portions having different Young's modulus that carry a stressed interface when assembled.

FIG. 15A is a perspective cut-away view of a lens capsule with a polymer monolith corresponding to the invention implanted therein.

FIG. 15B is a sectional view of the implant of FIG. 15A.

FIG. 15C is a sectional view of an alternative implant similar to that of FIGS. 15A-15B.

FIG. 16A is the cross-sectional shape of a natural lens capsule.

FIG. 16B is the cross-sectional shape of an implant monolith corresponding to the invention wherein the peripheral and posterior contours match a natural lens capsule.

FIG. 17 is the perspective sectional view of an adaptive optic monolith corresponding to the invention wherein the anterior optic surface is substantially recessed.

FIG. 18 is an illustration of a periphery of the adaptive optic monolith of FIG. 17 depicting the complete elasticity of the monolith's surface that engages the lens capsule.

FIG. 19A is an illustration of the haptics of a prior art lens that does not exhibit any elasticity at its surface that engages the lens capsule.

FIG. 19B is an illustration of another prior art haptics that does not exhibit any elasticity at its surface that engages the lens capsule.

FIG. 20 is the perspective sectional view of an adaptive optic monolith corresponding to the invention fabricated of first and second elastomer blocks.

FIG. 21 is the perspective sectional view of an alternative optic monolith that is fabricated of first and second elastomer blocks with a negative spherical aberration.

FIGS. 22A-22B are sectional views of an alternative optic monolith that is fabricated of first and second elastomer blocks with at least one rotational higher modulus block.

FIGS. 23A-23B are sectional views of an alternative optic monolith that is fabricated of first and second elastomer blocks with at an interface that carries features for enhancing deformation of the lower modulus block.

FIG. 24 is a perspective sectional view of an alternative optic monolith that has a stressed interface between first and second elastomer blocks.

FIG. 25 is the perspective sectional view of an alternative optic monolith that includes actuator features for deforming the surface of a low modulus central optic portion.

FIGS. 26A-26B are perspective sectional views of an elastomeric lens similar to the lens of FIG. 20 with a displaceable fluid in a deformable annular interior chamber with FIG. 26A showing the lens in an equilibrium accommodated shape and FIG. 26B showing the lens in a disequilibrium disaccommodated shape wherein the displaced fluid flattens the periphery of the optic portion.

FIGS. 27A-27B are perspective sectional views of a lens similar to the lens of FIGS. 26A-26B with an index-matched fluid in first and second interior chambers with FIG. 27A showing the lens in an equilibrium accommodated shape and FIG. 27B showing the lens in a disequilibrium disaccommodated shape.

FIGS. 28A-28B are perspective sectional views of a multi-component intraocular lens with anterior and posterior lens components with a displaceable index-matched fluid in an interior chamber of the negative power posterior lens.

FIG. 29 is a sectional view of an elastomer accommodating lens with a posterior lens surface having a flattened plane or concave plane for directing deformation forces on the posterior lens capsule caused by vitreous displacement.

### DETAILED DESCRIPTION OF THE INVENTION

In one embodiment, the implants of the invention is adapted to provide a biomimetic lens capsule complex that will enable actuation of an accommodative lens body between first and second powers, wherein the implant can have several variants. The objective of the invention is to provide a responsive implant body that when engaged with the capsular sac will mimic the inherent elastic response of a still accommodative lens capsule for cooperating with the ciliary muscles contraction-relaxation and vitreous displacement to alter the shape and power of an adaptive optic (e.g., a lens portion with a controlled deformable surface).

The biomimetic lens complex is provided by combining the lens capsule with a polymer implant body that engages at least the periphery of the capsule. The typical embodiments comprise, at least in part, a resilient polymer body that engages the periphery of a lens capsule and a central optic portion of a very low modulus polymer, optionally with a fluid-filled chamber, that is highly deformable or displaceable to alter the shape of the optic portion. In one embodiment, the implant is made in part from a class of shape memory polymer. The term “shape memory” has a meaning in the context of SMPs that differs from the more common use of the term uses in superelastic shape memory alloys, i.e., nickel titanium alloys. As background, a shape memory polymer is said to demonstrate shape memory phenomena when it has a fixed temporary shape that can revert to a “memory” shape upon a selected stimulus, such as temperature. A shape memory polymer generally is characterized as defining phases that result from glass transition temperatures in segregated linear block co-polymers: a hard segment and a soft segment. The hard segment of SMP typically is crystalline with a defined melting point, and the soft segment is typically amorphous, with another defined transition temperature. In some embodiments, these characteristics may be reversed together with glass transition temperatures and melting points.

In one embodiment, when the SMP material is elevated in temperature above the melting point or glass transition temperature of the hard segment, the material then can be formed into a memory shape. The selected shape is memorized by cooling the SMP below the melting point or glass transition temperature of the hard segment. When the shaped SMP is cooled below the melting point or glass transition temperature of the soft segment while the shape is deformed, that (temporary) shape is fixed. The original shape is recovered by heating the material above the melting point or glass transition temperature of the soft segment but below the melting point or glass transition temperature of the hard segment. (Other methods for setting temporary and memory shapes are known which are described in the literature below). The recovery of the memory original shape is thus induced by an increase in temperature, and is termed the thermal shape memory effect of the polymer. The temperature can be at or below body temperature (37° C.) or a selected higher temperature.

Besides utilizing the thermal shape memory effect of the polymer, the memorized physical properties of the SMP can be controlled by its change in temperature or stress, particularly in ranges of the melting point or glass transition temperature of the soft segment of the polymer, e.g., the elastic modulus, hardness, flexibility, permeability and index of refraction. The scope of the invention of using SMPs in in-the-capsule implants extends to the control of such physical properties, particularly in elastic composite structures described further below.

Examples of polymers that have been utilized in hard and soft segments of SMPs include polyurethanes, polynorborenes, styrene-butadiene co-polymers, cross-linked polyethylenes, cross-linked polycyclooctenes, polyethers, polyacrylates, polyamides, polysiloxanes, polyether amides, polyether esters, and urethane-butadiene co-polymers and others identified in the following patents and publications: See, e.g., U.S. Pat. No. 5,145,935 to Hayashi; U.S. Pat. No. 5,506,300 to Ward et al.; U.S. Pat. No. 5,665,822 to Bitler et al.; and U.S. Pat. No. 6,388,043 to Langer et al. (all of which are incorporated herein by reference); Mather, “Strain Recovery in POSS Hybrid Thermoplastics,” Polymer 2000, 41(1), 528; Mather et al., “Shape Memory and Nanostructure in Poly(Norbonyl-POSS) Copolymers,” Polym. Int. 49, 453-57 (2000); Lui et al., “Thermomechanical Characterization of a Tailored Series of Shape Memory Polymers,” J. App. Med. Plastics, Fall 2002; Gorden, “Applications of Shape Memory Polyurethanes,” Proceedings of the First International Conference on Shape Memory and Superelastic Technologies, SMST International Committee, pp. 115-19 (1994); Kim, et al., “Polyurethanes Having Shape Memory Effect,” Polymer 37(26):5781-93 (1996); Li et al., “Crystallinity and Morphology of Segmented Polyurethanes with Different Soft-segment Length,” J. Applied Polymer 62:631-38 (1996); Takahashi et al., “Structure and Properties of Shape-memory Polyurethane Block Copolymers,” J. Applied Polymer Science 60:1061-69 (1996); Tobushi H., et al., “Thermomechanical Properties of Shape Memory Polymers of Polyurethane Series and Their Applications,” J. Physique PIV (Colloque C1) 6:377-84 (1996)) (all of the cited literature incorporated herein by this reference). Also, see Watt A. M., et al., “Thermomechanical Properties of a Shape Memory Polymer Foam,” available from Jet Propulsion Laboratories, 4800 Oak Grove Drive, Pasadena Calif. 91109 (incorporated herein by reference). SMP foams function in a similar manner as the shape memory polymers described above. The scope of the invention extends to the use of SMP foams for use in any elastic composite structures, for example the peripheral portion of the implant that does not need to be optically transparent.

Other derivatives of SMPs that fall within the scope of the invention fall into the class of bioerodible shape memory polymers that again may be used in an elastic composite implant. As will be described below, one embodiment of capsular shaping element may be designed with composite portions that define a first modulus of elasticity and shape for a period of time after implantation to better engage with capsular sac (e.g., under cyclopegia) followed by a second modulus of elasticity and memory shape following a selected time period to provide a lower modulus adaptive optic portion.

In all variants of in-the-capsule implants described herein, the principal objectives relate to the design of an implant that will impart to the implant/capsular sac complex, an unstressed more spherical shape with a lesser equatorial diameter when zonular tension is relaxed, and a stressed flatter shape with a greater equatorial diameter in response to zonular tensioning forces. The resilient implant will provide the ability to absorb known amounts of stress-and release the energy in a predetermined manner in millions of cycles over the lifetime of the implant in cooperation with an optic that will provide variable focus.

In one embodiment, the biomimetic polymer implants corresponding to the invention define a first body portion or block that is adapted to engage the interior periphery of a capsular sac in 360°. Most often, this peripheral body comprises a non-optic portion of the lens system and imparts the desired memory shape to the biomimetic lens capsule. Most important, the peripheral implant body play roles as (i) a force transduction mechanism and as (ii) an actuation mechanism to “actuate” the central adaptive optic portion of the lens system—which comprises a second block or body portion of a low-modulus displaceable polymer or a body portion that carries a displaceable fluid media. In many preferred embodiments, the first and second (i.e., “actuator” and “actuated”) portions of the lens systems are blended seamlessly together. In some other embodiments, the first and second (actuator and actuated) portions of the lens system are independent components that mate after being independently introduced into the lens capsule.

In exemplary embodiments, the peripheral implant body is, at least in part, fabricated of a selected higher modulus polymeric material that imparts resiliency and memory shape to the capsule. The central adaptive optic portion of the implant is, at least in part, includes a lower modulus polymeric material and optional flowable media that will allow for substantial amplitude of deformation or accommodation in response to forces transduced by the peripheral body portion from physiologic forces (e.g. zonular excursion and vitreous displacement). In one embodiment corresponding to the invention, the lens body defines an “interface” between the higher and lower modulus portions—the “actuator” and “actuated” portions. The interface, when provided in an on-axis, rotationally symmetric shape can be designed to amplify force transduction between the block portions and thus enable high amplitude adaptive optic designs. In another embodiment corresponding to the invention, a plurality of “interfaces” are provided between higher and lower modulus, index-matched materials to gain mechanical advantage for controllably deforming the optic shape with limited transduced forces. In exemplary embodiments, the plurality of “interfaces” between higher and lower moduli portions of the optic is diffused to provide, if effect, gradients in the modulus across regions of the adaptive optic. Thus, the scope of the invention, in one embodiment, includes apparatus and methods that provide a high amplitude adaptive optic that has a memory shape and an on-axis, rotationally symmetric anisotropic stiffness, or architecture modulus, that transduces limited physiologic forces into amplified deformation forces applied to at least one deformable lens surface.

In any of the adaptive optic designs, all or part of the lens can be fabricated of a shape memory polymer as described above to provide for a compacted cross-section for introduction, or for post-implant adjustment of a parameter of a selected portion of the lens, for example, to adjust the modulus of a selected region, alter non-optic surface morphology or the like.

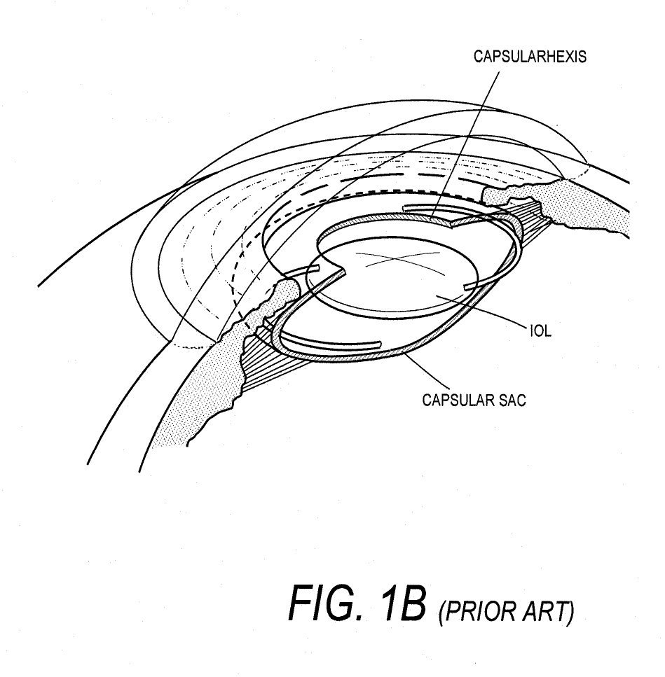

To understand principles underlying the some embodiments of the invention relating to the memory shape of an elastomeric monolith, “interfaces” as defined herein between the elastomer blocks and gradients in Young's modulus across the elastomer monolith, the cartoons of FIGS. 2A-14B are provided for purposes of explanation.

In FIGS. 2A and 2B, an elastomeric monolith 40 of a homogenous composition (uniform elastic modulus M) is depicted in a memory shape having an anterior surface contour indicated at 50A. By the term memory shape, it is meant that the monolith has an equilibrium or repose state and shape, wherein the monolith is deformable to a temporary, disequilibrium, tensioned or stressed state and shape. FIG. 2B illustrates that hypothetical uniform tension applied at first and second 180° opposed ends of the monolith about the “X” axis will cause a continuous uniform deformation in the central region relative to the “Y” axis and “Z” axis, resulting in a deflected or deformed surface contour indicated at the dashed line 50B.

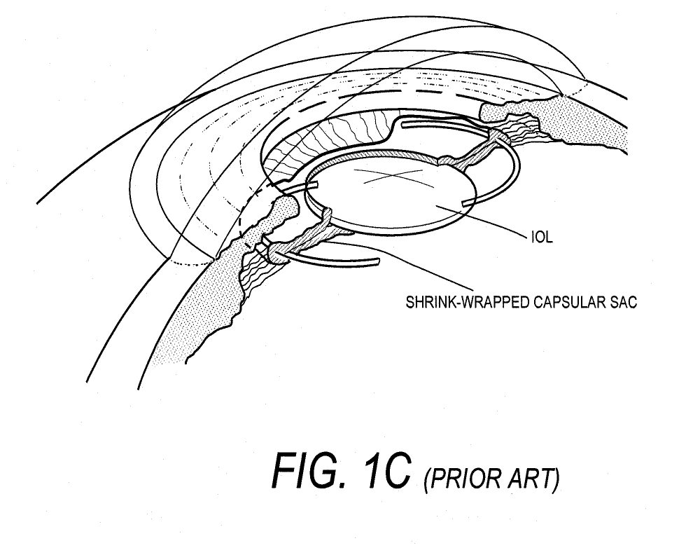

In FIGS. 3A and 3B, the elastomeric monolith 40 has an anisotropic modulus and is first depicted in a memory shape with anterior surface contour 50A. In this monolith, the peripheral end block portions have a first higher modulus M₁ and the central block portion has a second lower modulus M₂. The first and second block portions define interfaces 55 therebetween. Upon application of deforming forces about the “X” axis, it can be understood in FIG. 3B that amplified deformation of the displaceable, low modulus central portion will occur resulting in deformed surface curvature 50B. The deformation will be greater in FIG. 3B than the deformation which occurred in the uniform modulus monolith of FIG. 2B, all other parameters being equal, due to the displaceable, low modulus material or media M₂.

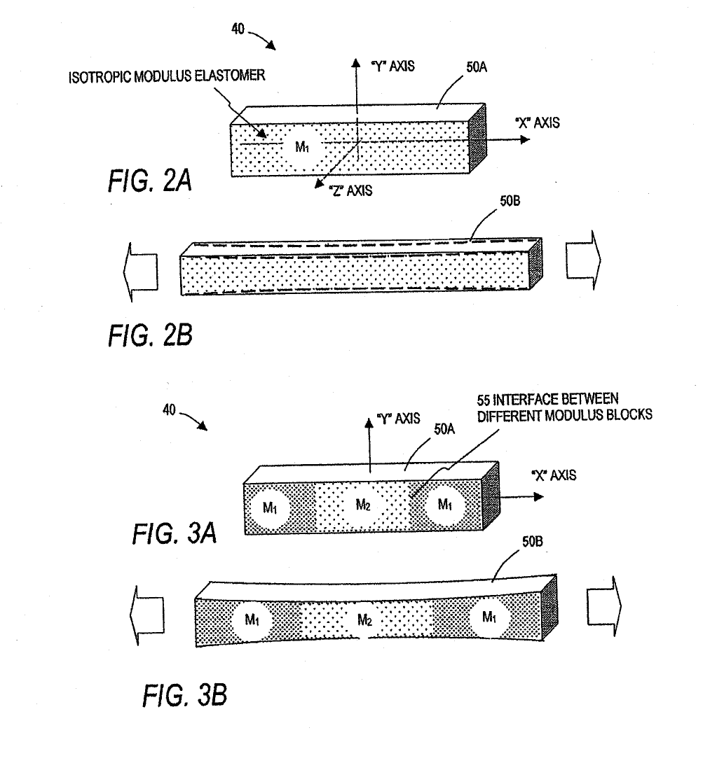

FIGS. 4A and 4B illustrate elastomeric monolith 40 with an anisotropic modulus that differs when compared to the body of FIGS. 3A-3B. In FIG. 4A, the higher modulus block M₁ is in the center with lower modulus blocks M₂ at the periphery—with interfaces indicated at 55 between the blocks. Upon application of deforming forces about the “X” axis, FIG. 4B depicts that the amplified deformation of surface curvature 50B occurs in the lower modulus portions M₂.

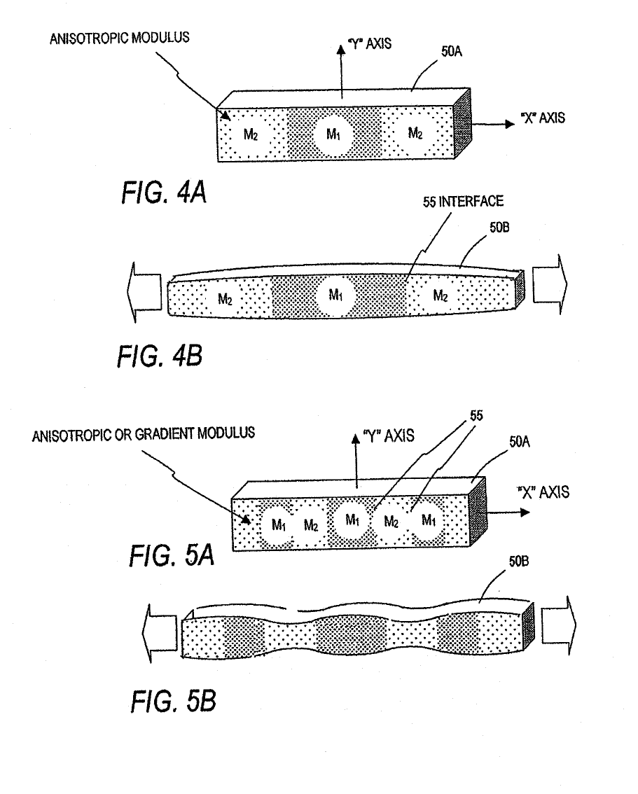

In FIGS. 5A and 5B, another elastomeric monolith 40 is depicted that illustrates more complex interfaces 55 between a plurality of higher and lower modulus blocks M₁ and M₂. In this monolith, the blocks and interfaces 55 are configured to control and redirect deforming forces generated about the “X” axis to amplify deflection in the orthogonal axes. Thus, the deformed surface contour 50B can be provided with a plurality of radii.

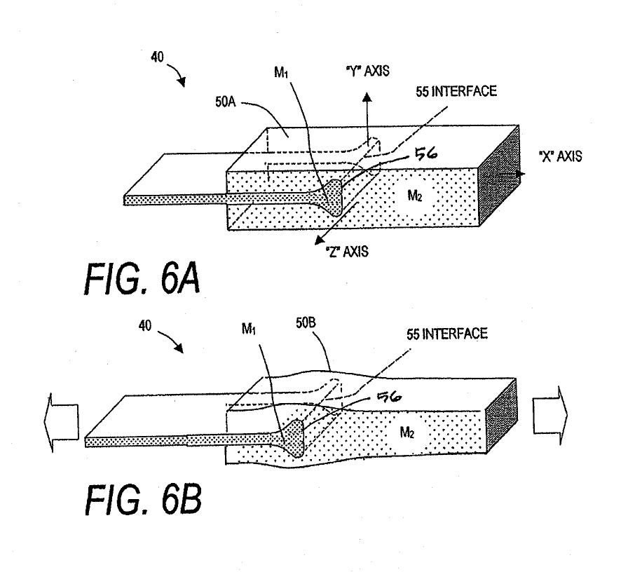

FIGS. 6A and 6B illustrate elastomeric monolith 40 that comprises a first higher modulus block M₁ that can be considered an “actuator” block that extends about the periphery, or within the interior, of a displaceable media or low modulus block M₂. In this embodiment, the interface 55 between the blocks defines shape means for enhancing deflection of the lower modulus block when selected forces are applied to the higher modulus block. In the monolith of FIG. 6A, it can be seen the interface shape defines a “cam” form 56 relative to the “X” axis along which forces may be applied. As can be seen in FIG. 6B, the application of deforming forces to the higher modulus block M₁ about the “X” axis would deform the lower modulus elastomer block M₂ generally as depicted in the amplified deformation of surface curvature 50B.

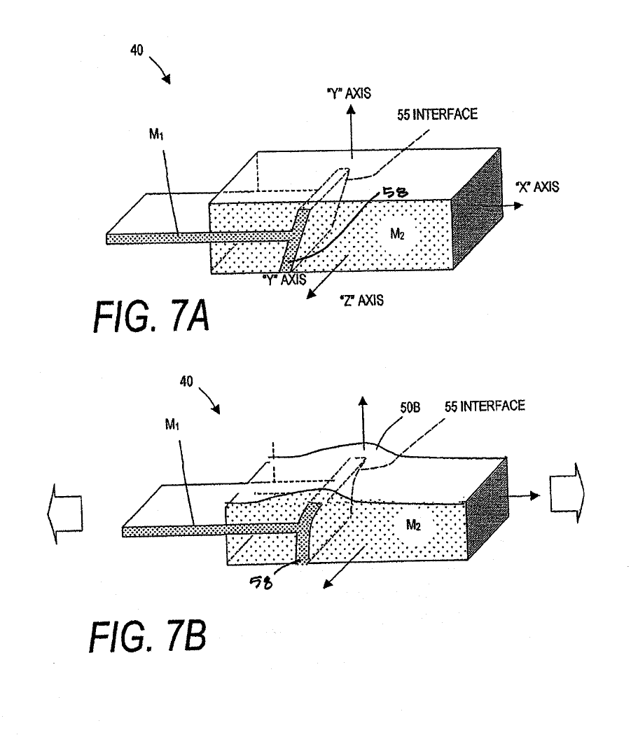

FIGS. 7A and 7B illustrate another elastomeric monolith 40 that again comprises a first higher modulus block M₁ that can be considered to function as an actuator block that extends within the interior of the displaceable polymer or low modulus block M₂. In this embodiment, the interface 55 between the blocks again defines shape means for enhancing deflection of the lower modulus block, wherein the interface shape defines a “lever” form 58 with a fulcrum. As can be seen in FIG. 7B, the application of forces to the higher modulus block M₁ about the “X” axis deforms the lower modulus elastomer block M₂ as depicted in deflected surface curvature 50B.

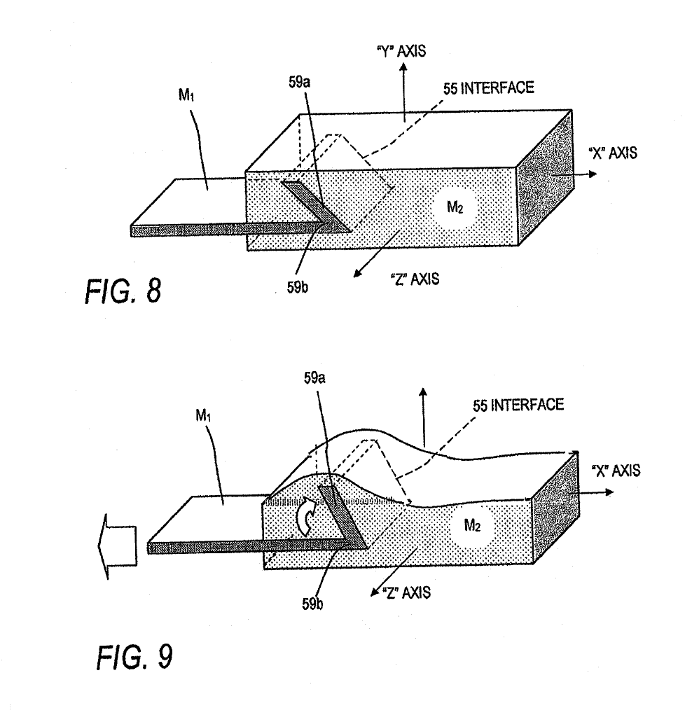

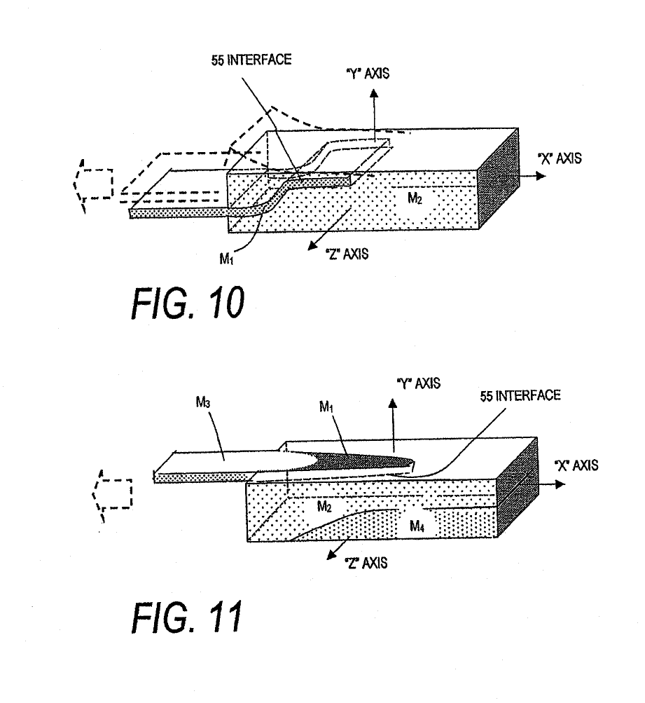

In another embodiment depicted in FIGS. 8 and 9, an actuator block includes at least one element 59a coupled with a hinge-like portion 59b to an actuator portion for creating leverage to deform surface curvature. This embodiment functions similarly to that of FIGS. 6A-6B and will enhance displacement of the displaceable low modulus material to deform the surface of the low modulus block M₂. In exemplary embodiments described above, the scope of the invention encompasses the fabrication of an elastomeric composition or body 40 (that optionally may carry an internal fluid-filled portion) that includes at least one interface between a first higher modulus elastomer block M₁ and a second lower modulus block M₂, wherein the shaped interface defines means for gaining a form of mechanical advantage for amplifying forces or re-directing the vectors of applied forces to thereby deform surface curvatures.

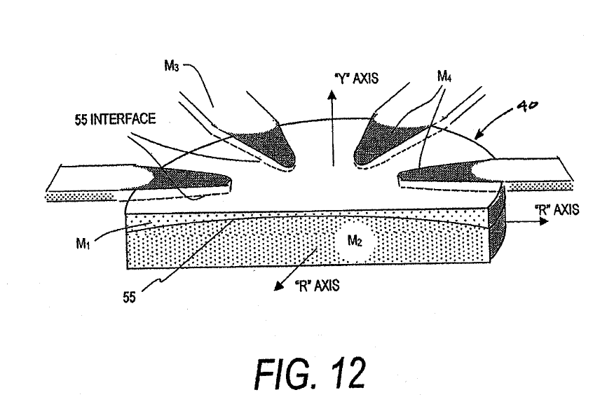

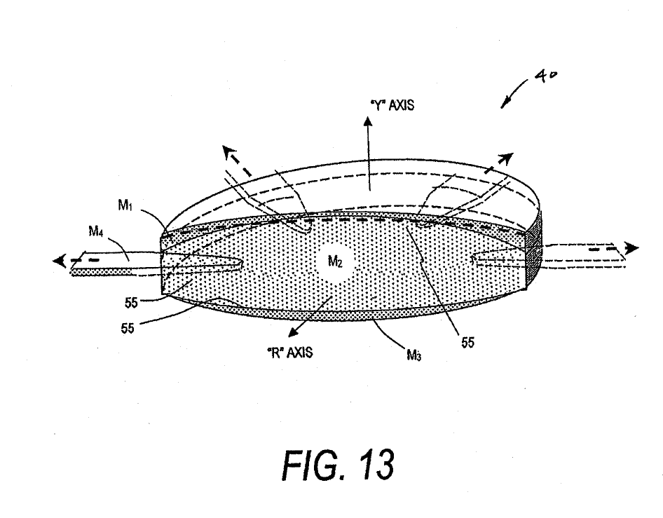

FIG. 10 depicts another elastomeric monolith 40 wherein the first higher modulus block M₁ can be a substantially thin body that extends as an actuator-like member within the interior of the displaceable, low modulus block M₂. In this embodiment, the first higher modulus block M₁ has a convoluted shape that will tend to straighten and thus controllably deform the second low modulus block M₂, generally as indicated at deflected surface curvature 50B in phantom view. FIG. 11 depicts an alternative elastomeric monolith 40 wherein the first higher modulus “actuator” block can have different moduli M₁ and M₃, or a gradient modulus (not shown). In FIG. 11, it also can be seen that the lower modulus block can be fabricated of a plurality of different blocks, for example with moduli M₁ and M₄, or any gradient modulus.

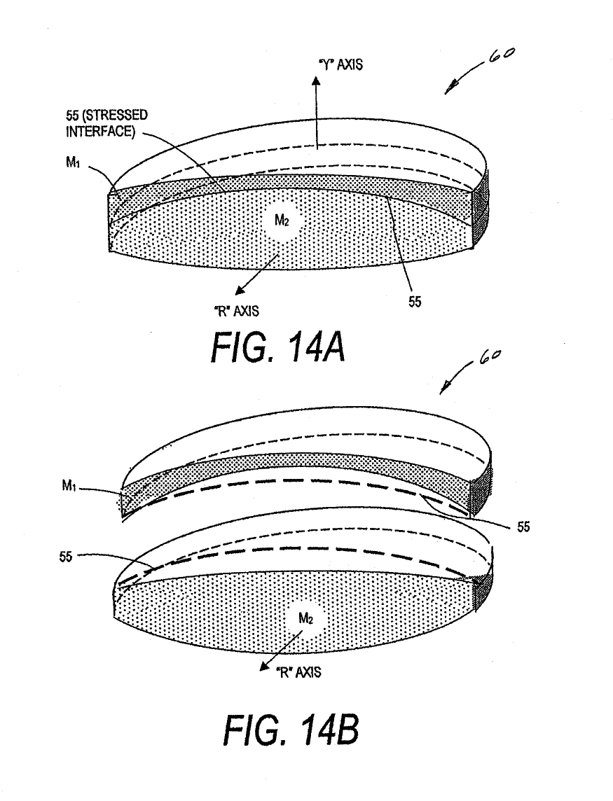

FIG. 12 depicts an embodiment wherein the elastomeric monolith 40 is more in the form of a lens wherein the varied moduli materials comprising blocks M₁ and M₂ that have a rotationally symmetric interface about a central “Y” axis. The scope of the invention includes a plurality of higher modulus “actuator” blocks M₃, of any of the types described above, that also are arranged in a radially symmetric manner.

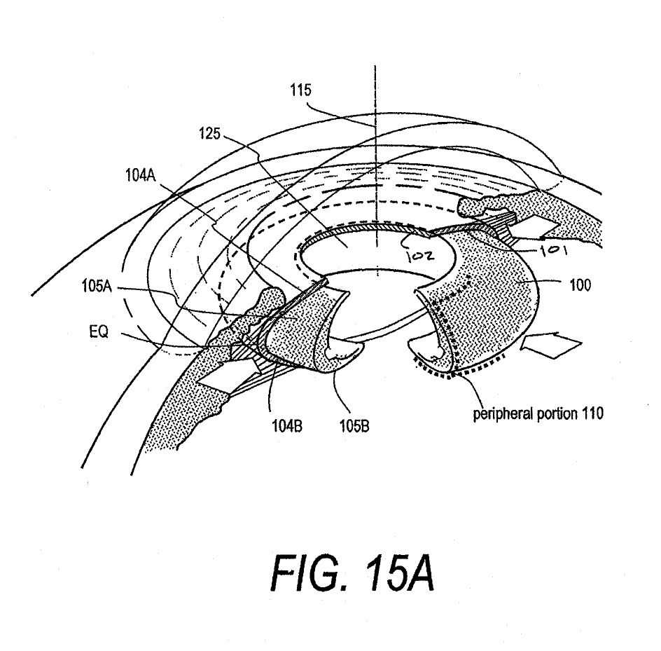

FIG. 13 depicts that an elastomeric monolith 40 that comprises a lens, or adaptive optic, for refracting light relative to the “Y” axis. An elastomer optic of the type in FIG. 13 can be inexpensively microfabricated. In this optic monolith, the lens body defines at least one rotationally symmetric interface 55 between controlled deformable surface layers M₁ and M₃ that have a higher modulus than the core portion of a low modulus displaceable media M₂. As can be seen in FIG. 13, the surface layers can have a rotationally symmetric variation in axial stiffness due to changes in thickness. In this embodiment, the anterior surface layer is thinner in the central region that would amplify deflection centrally. In the embodiment of FIG. 13, the posterior surface layer M₃ is thinner in the peripheral region that would amplify deflection peripherally. By this means of varying axial stiffness of a deformable surface layer M₁ or M₃, the optic can be induced to move between a first memory curvature and a second deflected temporary curvature. The surfaces can be configured to deform between selected radii or at least one such curvature can have a selected higher order spherical aberration. Spherical aberrations are the only higher order wavefront aberration that is rotationally symmetric—and in an adaptive optic corresponding to the invention can provide a lens for human lens capsule implantation that would define a selected negative spherical aberration at least in a temporary tensioned shape, and preferably the lens would define a selected negative spherical aberration in all shape between its memory shape and its lowest power temporary tensioned shape.

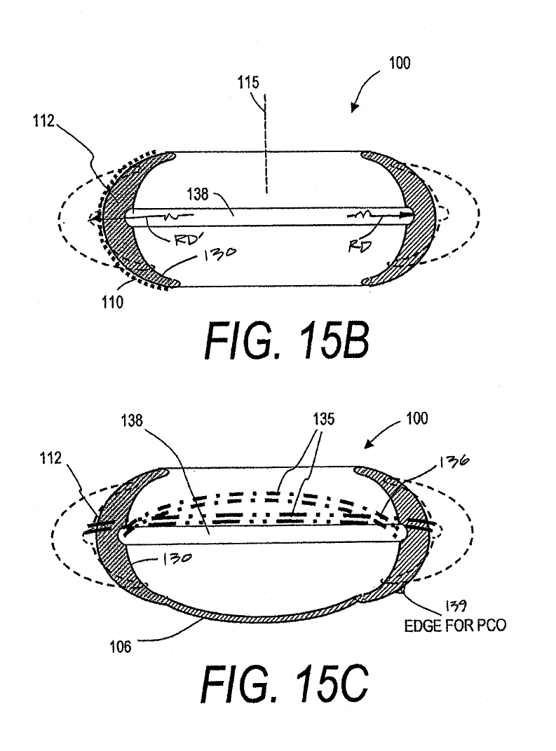

FIGS. 14A and 14B depict another elastomeric monolith 60 that comprises an adaptive optic for refracting light relative to the “Y” axis. The monolith of FIGS. 14A and 14B differs from the monoliths in FIG. 2A-13. In the earlier described deformable monoliths, the plurality of elastomer blocks have an equilibrium resting memory shape that is the same as when assembled into the monolith—which thus define the equilibrium memory shape of the monolith. In contrast, the elastomer monolith of FIGS. 14A and 14B is assembled of at least two elastomer blocks that have equilibrium resting memory shapes that are not in internal equilibrium when assembled and bonded together into a monolithic form. In other words, FIG. 14A depicts an assembled optic monolith that defines an “assembled” equilibrium resting memory shape, wherein at least one block portion is in a disequilibrium state and shape when assembled. FIG. 14B illustrates the two elastomer blocks in their memory equilibrium shapes when de-mated. It can be seen that interface 55 in the assembled monolith causes the deformation of the mating surfaces of the blocks. Thus, the adaptive optic of FIG. 14A has at least one interface that defines a stress continuum between the at least two block portions for adapting the shape of the lens in response to applied forces. This type of monolith with built in internal stresses about the at least one interface can assist is providing an actuation mechanism for applying deformation forces from a first block to a second elastomer block. By this means of providing bonded together lenses of different stressed and unstressed body portions M₁ and M₂, the assembled lens body can be made respond to very low levels of force to move the lens body between different shaped and powers.

The above description assists in describing a method corresponding to the invention for designing, optimizing and making an elastomeric adaptive optic. The method consists of the steps of (i) providing the shape of a lens in a memory shape wherein the polymer has a uniform Young's modulus or optionally carries a fluid filled chamber; (ii) modeling surface curvature deflection in response to equatorial forces on the lens with high resolution finite element analysis (FEA) means thereby producing data on deformation, and resistance to deformation, at a plurality of FEA elements; (iii) computing Young's modulus variation required at said plurality of FEA elements to provide a selected surface curvature deflection thereby providing an optimum hypothetical lens design; and finally (iv) calculating by FEA means an actual lens design that emulates said hypothetical lens design while constraining said Young's modulus variation to a plurality of uniform modulus elastomer blocks of the monolith wherein said blocks define an on-axis, rotationally symmetric interface therebetween, or when the lens is deformed by radially symmetric actuators as described above.

In any of the embodiments above, the higher modulus block or actuator block can be of a shape memory polymer (SMP) that has a temporary strained shape that stores energy and a memory un-strained shape. Thus, the SMP block can be altered in response to any stimulus (e.g., light or hydration) to adjust in shape and apply forces to the low modulus block. The scope of the invention thus encompasses using energy stored within the monolith to adjust a parameter of the monolith, or the use of external forces to adjust the monolith.

In a method of the invention relating to intraocular adaptive optics lenses, the SMP component can be utilized to provide at the least the non-optic portion of the adaptive optic with a temporary shape that corresponds to a disaccommodated lens capsule. The lens is then implanted under pharmacologically- induced cyclopegia as is known in the art. This effect can last from 24 hours to about a week to allow for tissue in-growth into a porous periphery of the lens body to better couple the implant with the lens capsule. Thereafter, the lens body returns to its memory shape that corresponds to the form of a natural accommodated lens capsule. The lens body can return to its memory shape by means of any selected stimulus that alters the SMP component, such as ambient light over time, light energy from a laser or other source or hydration.

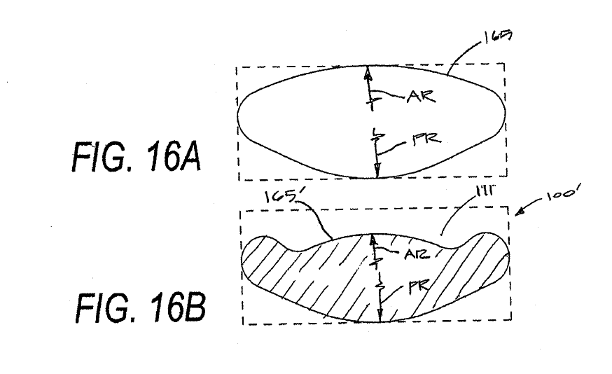

FIGS. 15A-20 depict polymer monoliths for implantation in a lens capsule for imparting biomimetic shape responses to the lens capsule. The monoliths fall into two classes: (i) implant bodies which engage at least the periphery of the lens capsule and cooperate with an independent drop-in replaceable optic, and (ii) implant bodies that carry an integrated central optic portion.

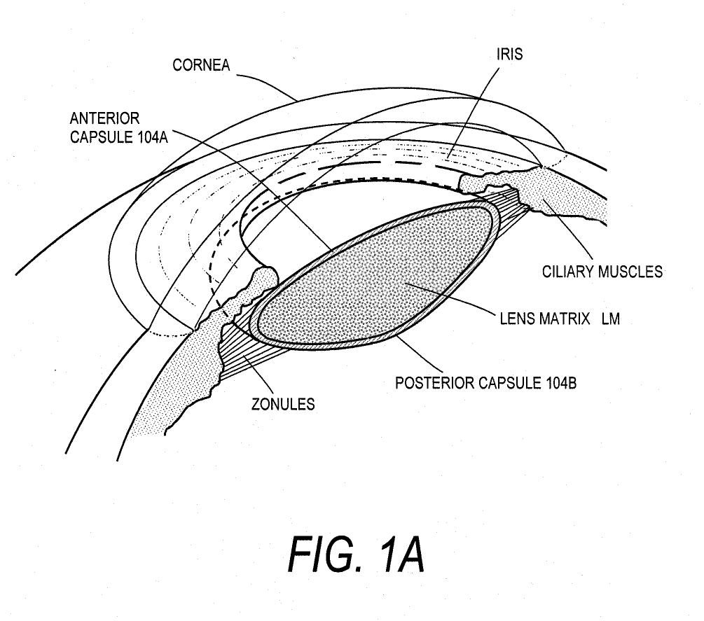

FIGS. 15A-15B illustrate sectional views of a resilient polymer implant body 100 corresponding to the present invention, with FIG. 15A depicting the implant body 100 in a lens capsule 101. In this disclosure, the term “lens capsule” refers to the thin membrane or structure that surrounds the lens matrix, i.e., its cortex and nucleus. In FIG. 15A, the capsule 101 has a capsularhexis 102 in the anterior capsule 104A for removal of the crystalline lens matrix LM. (FIG. 1A). The posterior capsule is indicated at 104B. This disclosure will adopt the terminology in common use by ophthalmologists that defines the anterior capsule as the portion of the capsule that is anterior to the capsular equator region EQ, and the posterior capsule as the portion that is posterior to the equatorial region. In FIGS. 15A and 15B, the implant body 100 generally can be defined as having anterior surface region 105A that engages the post-capsularhexis anterior capsule 104A and a posterior surface region 105B that engages at least a portion of the posterior capsule 104B. In another embodiment, as can be seen in FIG. 15C, the posterior surface region 105B is continuous in central region 106 and extends across the entire central region of posterior capsule 104B. In FIG. 15C, the central region 106 of the implant is depicted as a zero power element, but also can be a positive or negative power element as will be described below.

Referring to FIG. 15A, the implant body 100 also defines a peripheral or circumferential surface region indicated at 110 that engages the inner equatorial surfaces of the capsular sac in 360°. As can be seen in FIG. 15A and 15B, the peripheral surface region 110 also is defined as transitioning somewhat over that anterior and posterior surface region 105A and 105B as the surface extends radially inward. As described below, this peripheral surface region 110 thus provides the three-dimensional implant body portion that thus impart “shape” to the lens capsule. It is the combination of this implant body portion 110 and the capsule 101 that provides a biomimetic lens capsule that cooperates with the eye's natural accommodation mechanisms to enable new classes of accommodating lens systems. This implant-capsule complex can thus mimic a naturally accommodative human lens capsule in force transduction. Further, the isovolumic implant body 100 provides for peripheral shape changes between its equilibrium memory shape and temporary stressed, disequilibrium shapes to impart to sac its natural peripheral shape. FIG. 15B illustrates the implant body 100 in its memory accommodated shape with a phantom view (dashed line) of the body 100 in a stressed, disaccommodated shape.

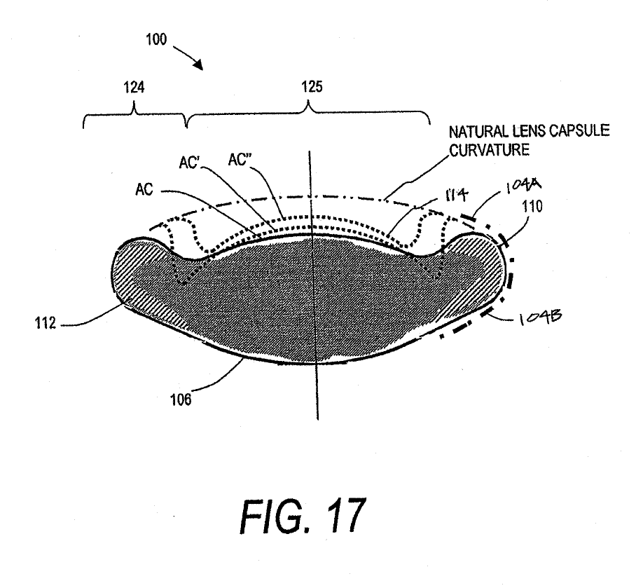

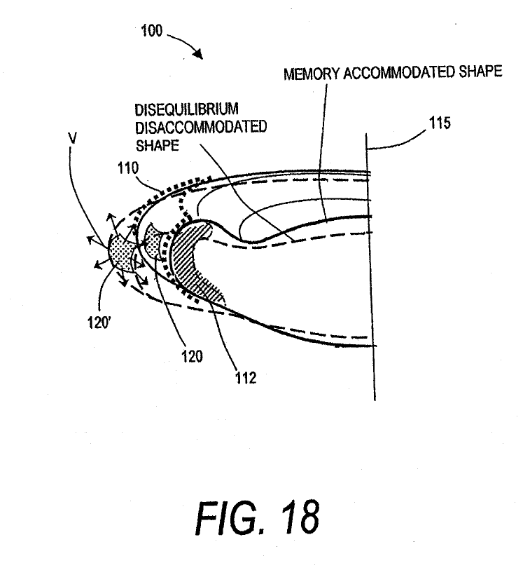

FIG. 16A depicts the shape of a natural lens capsule in an accommodated state with the anterior curvature radius AR in the range of 10 mm. The posterior curvature radius PR in the range of 6 mm. FIG. 16B illustrates a sectional shape of one embodiment of a lens implant 100 with a recessed anterior portion 111 wherein the anterior radius AR again is in the range of 10 mm and the posterior radius PR in the range of 6 mm—the same as in FIG. 16A. FIG. 17 illustrates an exemplary range of shapes (anterior curvatures AC, AC′ and AC″) that fall within the scope of the invention wherein the periphery 110 of the implant monolith 100 and the posterior surface 106 have the same shape and radii as a natural lens capsule. In FIG. 17, the central lens is within a recess which has a continuous 360° smooth arcuate transition from the non-optic optic portion 124 to the optic portion 125 to prevent unwanted reflections, wherein the multiple radii of the transition are from about 0.01 mm to 10 mm, or from about 0.05 mm to 5 mm. The recess of the anterior lens surface from that anterior-most plane of the lens can have a depth of at least 1 mm, 2 mm, 3 mm and 4 mm.

Of particular interest, an optic monolith 100 of the invention in FIGS. 16B and 17 has a non-optic portion 124 with a central anterior optic region 125 that is from about 4.5 mm. to 7 mm. in diameter wherein the anterior optic curvature is recessed from the anterior-most plane of the peripheral or non-optic portion 124. By this means, the anterior curvature AC of the optic monolith portion can be steepened with a radius of less that 10 mm. More preferably, the deformable curvature AC in it memory shape has a radius of less that about 9 mm; and more preferably less than 8 mm. FIG. 17 illustrates that there are a range of surface curvatures about the implant periphery and anterior surface 114 that transition to the recessed optic portion and the deformable anterior curvature AC.

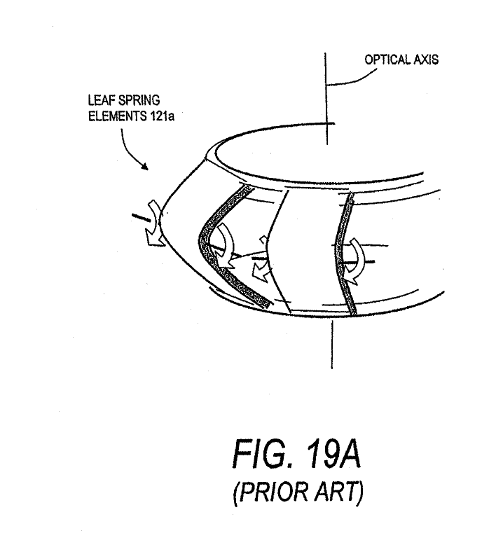

As will be described below, the implant and method of the invention (whether the implant cooperates with a drop-in lens or comprises an integrated optic monolith): (i) provide a resilient elastomeric monolith wherein 100% of the peripheral body portion and surface region 110 that comprises a force-application structure to apply forces to the capsular sac 101 to return the implant-capsule complex to its accommodated shape; (ii) provide a body having a resilient deformable-stretchable surface 110 that applies forces to the capsule in a continuum of surface vectors indicated at V in FIG. 18 about 360° of the inner surface of the capsular relative to axis 115. In FIG. 18, a selected hypothetical area 120 of the surface region 110 is shown as it deforms in multiple surface vectors to stressed condition 120′, with vectors V indicating the stress and restorative forces about the boundary between the implant and capsule 101 (see FIG. 18).

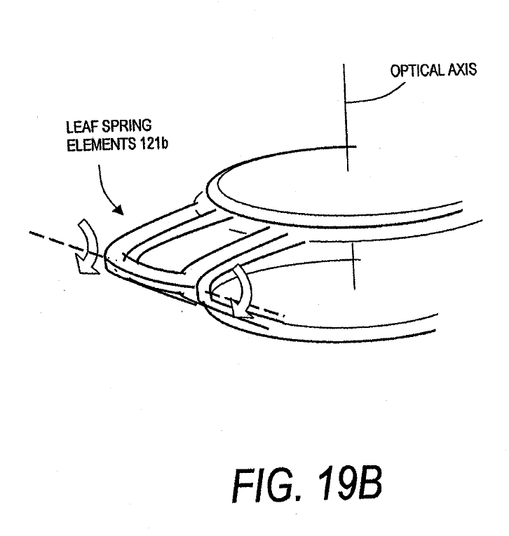

Of particular interest, this aspect of the present invention distinguishes the implant body 100 from other IOL implants in the patent literature that attempt to prop open the capsule 101. In the patent literature and published patent disclosures, the prior art designs simply use one form or another of “leaf spring” type members that bend or flex about an apex within the sac to deliver 2D forces as indicated in FIGS. 19A and 19B. The IOL designs in the patent literature and FIGS. 19A and 19B by their very nature must “slip” within the lens capsule 101 and thus cannot optimize force transduction from the zonules through the capsule to the implant. None of the IOL designs in the patent literature has an implant body with a 360° continuous peripheral engagement surface for providing force application means to apply restorative forces in a continuum of surface vectors V (FIG. 18) to insure that slippage about the implant boundary cannot occur. The illustration of FIG. 19A depicts the leaf-spring type haptics 121a of the lens designs of Woods in U.S. Pat. Nos. 4,790,847; 6,217,612; 6,299,641; and 6,443,985, and Sarfarazi in U.S. Patent Publication Nos. 20040015236; 20030130732; and U.S. Pat. Nos. 6,423,094 and 6,488,708. The illustration of FIG. 19B depicts the leaf-spring type haptics 121b of Zadno-Azizi et al in U.S. Patent Publication No. 20030078657; and U.S. Pat. Nos. 6,899,732; 7,087,080; 6,846,326; 7,226,478; and 7,118,596. The factor of slippage, at the scale of the implant and the limited forces that must be captured and transduced by the system, is a large component in a force transduction system for providing a high amplitude accommodating lens.

As defined herein, referring to FIG. 17, an exemplary optic monolith 100 has a peripheral non-optic portion 124 and a central optic region indicated at 125 that ranges from about 4.5 to 7.0 mm in diameter. In this disclosure, the term axis and its reference numeral 115 are applied to both the natural lens capsule and the implant body 100, and the term axis generally describes the optical axis of the vision system. The axial dimension AD refers to the dimension of the capsular implant or implant/lens capsule complex along axis 115.

A principal objective of the invention is optimizing force transduction, by which is meant that the implant is designed to insure that zonular tensioning forces are transduced (i) from the zonules to the sac, (ii) from the sac to the implant surface region 110 without slippage, and (iii) from the surface region 110 to an adaptive optic body within the central optic region 125 of the body. It is a further objective to insure that ciliary muscles, zonules and the entire physiologic accommodative mechanism, including vitreous displacement, remains intact and functional throughout the life of the patient. To accomplish these objectives, the implant body 110 is dimensioned to match the natural lens capsule to thus preserve zonular connections to the sac, which in turn it is believed will optimally maintain ciliary muscle function. Referring again to FIGS. 15A and 15B, one embodiment of implant body 100 has a peripheral surface region 110 that in its memory shape in 360° corresponds to the shape of natural, young accommodated lens capsule (FIG. 15A). Likewise, in its temporary stressed shape under zonular tensioning (FIG. 15B), the implant body 100 defines a controlled shape peripheral surface 110 in 360° that corresponds to the shape of natural disaccommodated lens capsule. By designing the memory and temporary shapes of the implant monolith to mimic a natural lens capsule in 360°, the zonular attachments can be preserved which, in turn, will optimize force transduction from the zonules to the capsule and to the implant body 100.

In FIG. 15A, the implant monolith 100 is shown in perspective view as it would appear in a memory, unstressed state which is dimensioned to provide the implant-capsular sac complex with the shape of a natural lens capsule, excepting the open anterior portion that corresponds to the capsularhexis. The implant of FIGS. 15A and 15B thus comprises an annular open-centered polymer body 100 with a peripheral surface region 110 that is dimensioned to engage the equatorial and peripheral portion of the capsular sac—and provide the capsule complex with the desired memory (accommodated) shape and stressed (disaccommodated) shape. The structure and modulus of the implant body 100 is described further below, but it can be understood that body 100 then can function and as actuation mechanism for deforming or actuating the shape of the central optic region 125 that is rotationally symmetric about axis 115. The implant body 100 provides the implant-capsule complex with the stress-absorbing and stress-releasing characteristics of a young, still accommodating lens, and also can adapt the shape and/or translation of a central lens.

As can be seen in FIGS. 15B and 15C, the inner surface 130 of body 100 is actuated in 360° between the radial dimensions indicated at RD and RD′. In one embodiment depicted in FIGS. 15B and 15C, the implant monolith 100 is adapted to receive a flexible drop-in accommodating IOL 135 shown in phantom view. In FIGS. 15B and 15C, the drop-in intraocular lens has peripheral haptics 136 that engage an engagement structure 138 such as groove or notch that may be annular or a plurality of cooperating features to engage haptics 136 of the lens 135. For example, two, three or four or more haptic elements 136 of IOL 135 can engage the annular engagement structure 138, by springably engaging the groove, or by locking into the groove by rotation or a spring-clip arrangement or any other lock-in structure. Alternatively, the engagement structure can carry means for photothermal or photochemical attachment of the peripheral haptics 136 with the engagement structure 138 by post-implant actuation with a light source.

Still referring to FIGS. 15B and 15C, the implant body 100 can be of any suitable polymer having a selected elastic modulus in the range between 1 KPa and 2000 KPa, or between 10 KPa and 500 KPa, or between 20 KPa and 200 KPa. In one embodiment, the polymer is a shape memory polymer that provides the desired modulus in its memory shape. In another embodiment, the adaptive implant has an anisotropic modulus and is fabricated as described previously. Thus, the adaptive implant body comprises an elastomeric body having a specified elastic modulus in its equilibrium memory shape that imparts to a capsular sac periphery its natural shape in an accommodated state. The body is deformable to a disequilibrium stressed shape in responsive to equatorial tensioning, the monolith capable of applying restorative forces of 1 to 3 grams to move the monolith toward the equilibrium shape from the disequilibrium shape. Further, elastomeric body has an interior structure wherein 100 per cent of a peripheral surface region of the monolith applies restorative forces to an interface with the capsular sac. In another embodiment, the adaptive implant has at least one exterior 360° edge feature 139 for posterior and/or anterior capsular opacification (FIG. 15C).

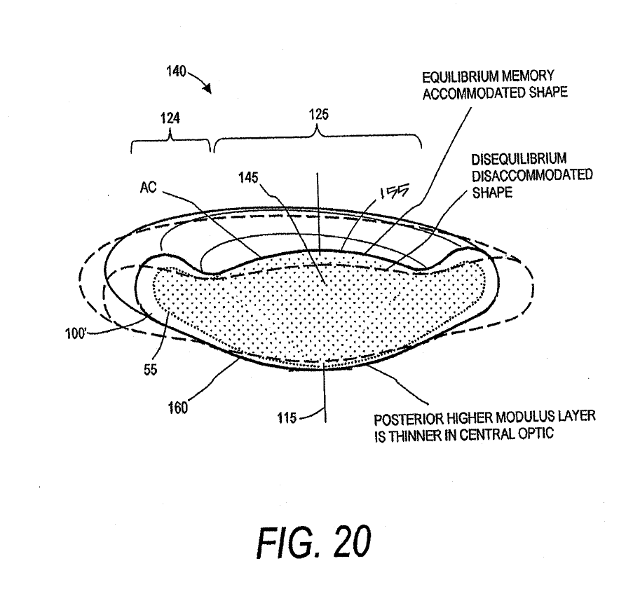

Now turning to FIG. 20, another embodiment of adaptive optic is depicted wherein the implant comprises an elastomer monolith 140 that defines two elastomer block or body portions. The peripheral block portion indicated at 100′ is substantially similar to implant body 100 of FIGS. 15A-15C. The second block portion 145 comprises an adaptive optic or lens for refracting light. The lens portion 145 is of an ultralow modulus polymer that can be “actuated” or “deformed” by the shape change of interface 55 between the blocks 100′ and 145—wherein interface 55 is as described above in the exemplary elastomer monoliths of FIGS. 2A through 14B. The interface 55 also can be compared with the inner surface 130 of implant body 100 as in FIGS. 15A-15C. In this embodiment, the lens portion 145 comprises a polymer having a selected elastic modulus that is less than 1000 KPa, or less that 500 KPa, or less that 200 KPa.

In FIG. 20, the adaptive optic block or deformable lens body 145 is symmetric for refraction of light relative to optical axis 115, and is depicted as positive power lens although negative power lenses fall within the scope of the invention to cooperate with a piggy-back or drop-in lens (see FIGS. 15B-15C). Of particular interest, this embodiment of monolith 140 defines at least one interior, on-axis, rotationally symmetric interface 55 between at least two elastomeric block portions, 100′ and 145, wherein each block portion has a different Young's modulus. It has been modeled that the peripheral block portions 100′ can function in effect as an actuator to cause substantial deformation of at least the anterior surface 155 of the lens. The elastomeric monolith has an equilibrium memory shape as in FIG. 20 and is deformable to the disequilibrium temporary shape (phantom view in FIG. 20), wherein the memory shape provides a selected focusing power and the disequilibrium temporary shape provides a lesser focusing power. The elastomeric optic monolith moves from its equilibrium memory shape to the disequilibrium temporary shape of FIG. 20 in response to equatorial tensioning forces as well as vitreous displacement. The adaptive system also can function to flex the posterior surface 160 of the lens, and for this reason the higher modulus material is preferentially thinned about the axis to induce central posterior surface steepening. In any of these embodiments, at least the peripheral block (or a portion thereof) can be fabricated of a shape memory polymer as described above. Thus, the non-planar on-axis, rotationally symmetric interface 55 between the two block portions is adapted for force transduction from the higher modulus block peripheral block 100′ to the lower modulus adaptive optic block 145. It should be appreciated that any of the feature and shapes for enhancing optic deformation (soft elastomer cams etc.) of FIGS. 4A-14 can be provided in the interface 55. The lens body can be substantially deformed by less than 3 grams of radial forces, or less than 2 grams of radial forces, or less than 1 gram of radial forces.

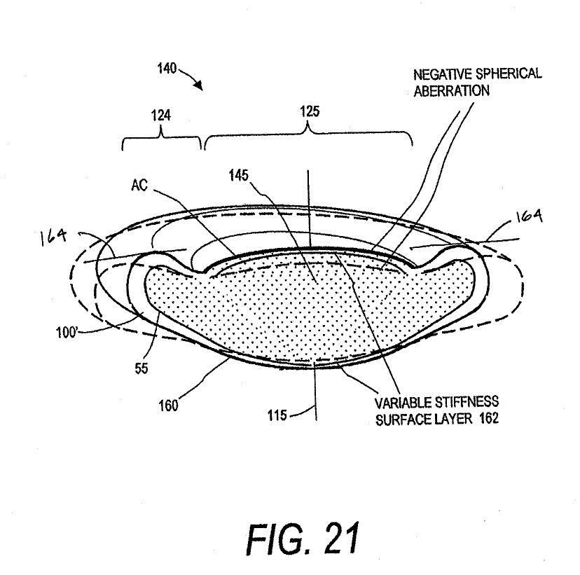

In one embodiment, either or both the anterior and posterior surfaces 155 and 160 provide the optic with a negative spherical aberration, at least in the stressed temporary shape of FIG. 21. This aspect of the invention provides a higher order aberration correction that has been found important for contrast sensitivity and is the only higher order aberration correction that is rotationally symmetric about the optic axis 115. Additionally, the lens can be provided with a controlled deformable-shape surface layer indicated at 162 in FIG. 21. The tensioned shape can be designed to provide deformable layer with the negative spherical aberration, or a selective surface modification could cause the same desired result. In other words, the anisotropic modulus or varied stiffness of the layer 162 can induce the negative spherical aberration in the controllably deformable surface. Adaptive optic algorithms developed for flex surface mirrors can be utilized to engineer anisotropic modulus or varied stiffness (flex characteristics) of the deformable surface or surfaces.

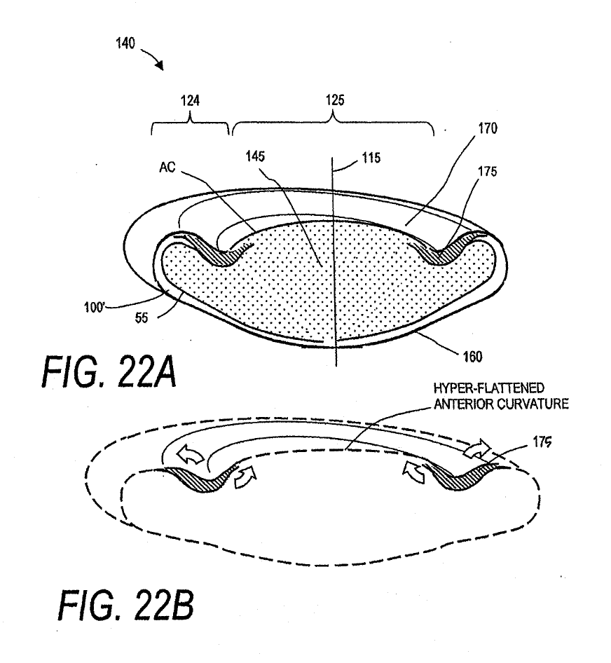

The embodiments of FIGS. 20-21 again illustrate that the anterior surface of the optic block 145 is recessed relative to the anterior-most plane 164 of the implant 140 in its memory shape. This allows for a hypersteepened curvature of anterior surface AC as described previously. If such a steep anterior curvature were provided in a continuous smooth curve 165 (FIG. 16A), it would impinge upon the iris and not function. Thus, the recessed anterior surface AC can provide a very steep curvature for increasing dioptic power. FIGS. 22A-22B depict a similar embodiment wherein the arcuate trough 170 about the anterior surface of low modulus lens block 145 can comprise a stiffened or higher modulus portion 175 that can “rotate” somewhat upon zonular excursion and equatorial tension to function as a lever means (cf. FIGS. 6A-7B) on the low modulus displaceable block 145 to axially lift and displace the polymer about the periphery of the optic portion 125 to thereby move the anterior surface from a steep curvature (FIG. 22A) to a highly flattened curvature (FIG. 22B). This effect would not be possible if the anterior surface of the lens had a smooth curvature—without the recessed anterior surface 165′ as in FIG. 16B. The scope of this aspect of the invention includes fabricating an intraocular lens dimensioned for implantation in a capsular sac, wherein an interior of the lens includes a displaceable media deforming the lens surface to provide first and second powers, and wherein the lens surface is configured for high amplitude axial deformation by the displaceable media about a periphery of the optic portion and configured for low amplitude axial deformation about the optical axis.

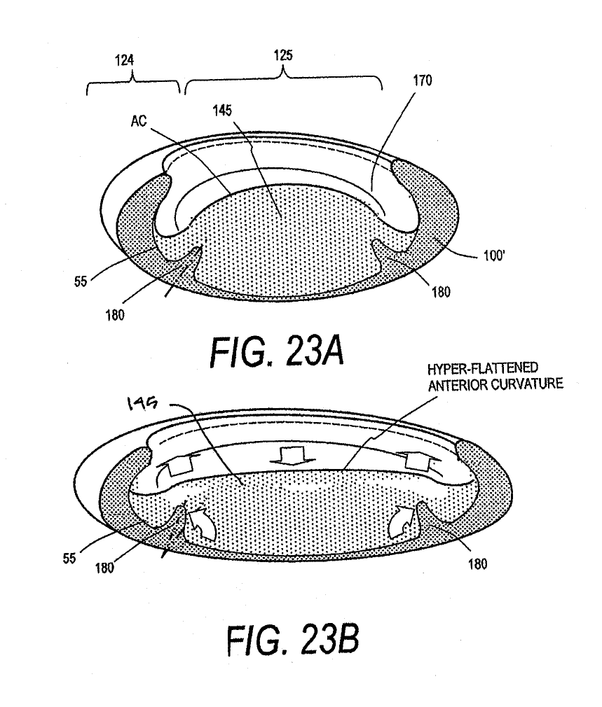

In another embodiment of FIGS. 23A and 23B, implant 140 comprises an elastomeric monolith again formed of a first monolith or block for transferring forces about interface 55 to the second monolith block to alter at least one property thereof, wherein the first and second monolith blocks, 100′ and 145, each have a different elastic modulus. In the embodiment of FIG. 23A, it can be seen that the interface 55 provides shape features for surface deformation and flattening about trough 170 wherein the features extend across a substantial interior region of the lens or project or extend into the interior of the low modulus optic block 145. Of particular interest, the elastomeric structure in its resting state provides at least one mechanical form having a rotationally symmetric structure that is adapted for gaining mechanical advantage in transferring forces to the second monolith block 145 from the first monolith block 100′. In other words, the mechanical form or forms 180 are capable of transferring forces at least in part from a first vector to an orthogonal vector—that is from an equatorial vector to a more axial vector. As can be seen in FIGS. 23A and 23B, the mechanical form 180 has the effect of a lever arm or cam in displacing the anterior surface AC of a peripheral region of the optic block 145 to cause hyper-flattening when under equatorial tensioning (FIG. 23B). In related embodiments, the elastomeric structure can have any mechanical form such as at least one of a lever, a fulcrum, a cam, a pivot or the like. In all the above embodiments, the elastomeric blocks within the central optic region are transparent index-matched elastomers. The embodiment of FIGS. 23A-23B can be compared to the structures of the cartoons of FIGS. 6A-9, all of which can be provided as a continuous feature about 360° of the interface 55 or can be provided as a plurality of about 30 to 100 radially spaced apart features in the interface 55 between the polymer blocks 100′ and 145.

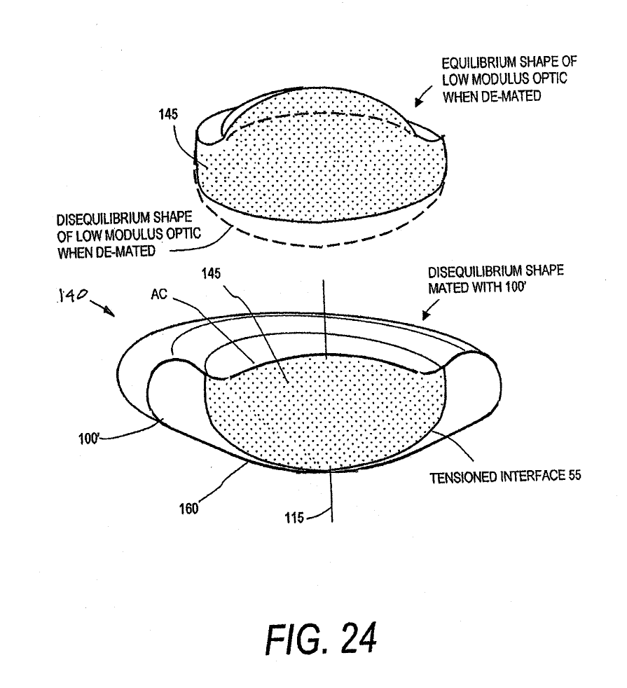

In another embodiment in FIG. 24, the adaptive optic 140 again comprises an elastomeric monolith having an optical axis 115 wherein the monolith defines at least one interior non-planar on-axis, rotationally symmetric interface 55 between at least two block portions (100′ and 145) wherein each portion has a different Young's modulus. In the embodiment of FIG. 24, the adaptive optic monolith has an equilibrium resting memory shape. Each block portion, if de-mated from one another, would have a resting memory or repose shape. However, the assembled and bonded-together blocks causes the lower modulus block 145 to be in a substantial disequilibrium or tensioned shape. In other words, the adaptive optic has at least one interface therein that defines a stress continuum between the at least two block portions for adapting the shape of the lens in response to applied forces. The monolith body then trends toward a bi-stable type of body—wherein a certain degree of zonular tensioning will deform block 100′ to certain extent and then the built in stress in optic block 145 will apply additional impetus to cause anterior curvature flattening. The scope of the apparatus and method of the invention includes providing and implanting an intraocular lens with first and second body portions (e.g., a peripheral lens body portion and a central lens body portion) that each have a repose state, and wherein the body portions are coupled so that one body portion is in a repose or equilibrium state while the other body portion is in a tensioned or disequilibrium state in the accommodated configuration and in the disaccommodated configuration. This system and method allow for very slight forces of zonular excursion and vitreous displacement to move the lens between the accommodated and the disaccommodated configurations.

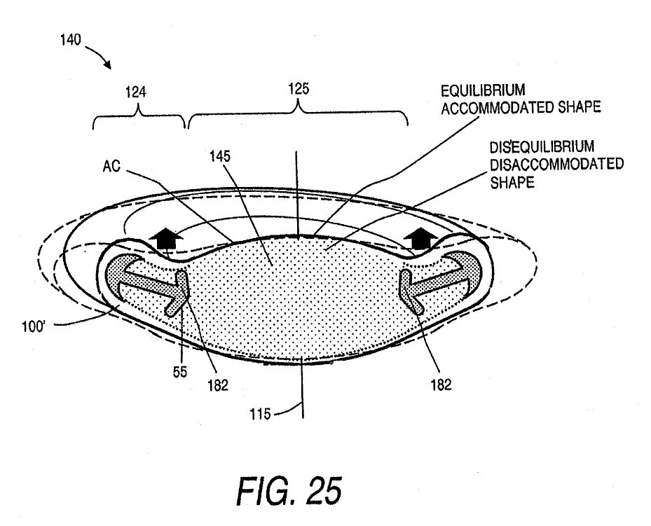

FIG. 25 depicts an alternative embodiment of deformable lens 140 that includes an interior block of a low modulus displaceable media 145 about optical axis 115. In this embodiment, the symmetric interface 55 between elastomeric block portions 100′ and 145 defines an actuator element 182 (or a plurality of radially spaced apart elements 182) that can have the aspects of any of the actuator elements shown in FIGS. 6A, 6B, 7A, 7B, 8 and 9. It can be understood that accommodating forces can cause movement of the actuator element or elements 182 to cause substantial deformation of at least the anterior curvature AC of the lens. Again, the arrows in FIG. 25 indicate that accommodating forces will cause substantial amplitude of deformation of the displaceable material 145 about of the periphery of the optic portion 125 and limited amplitude of deformation about the optical axis 115.

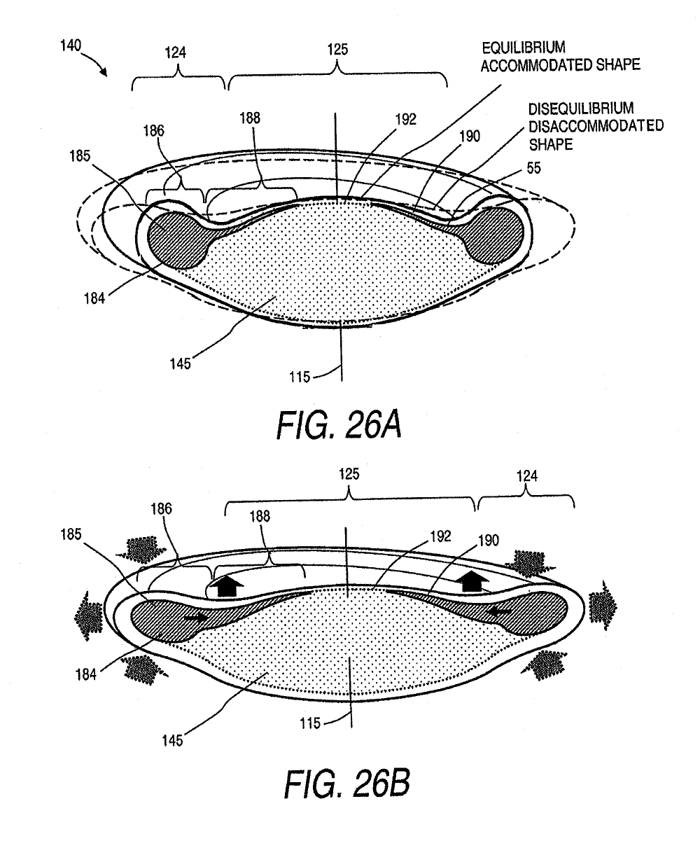

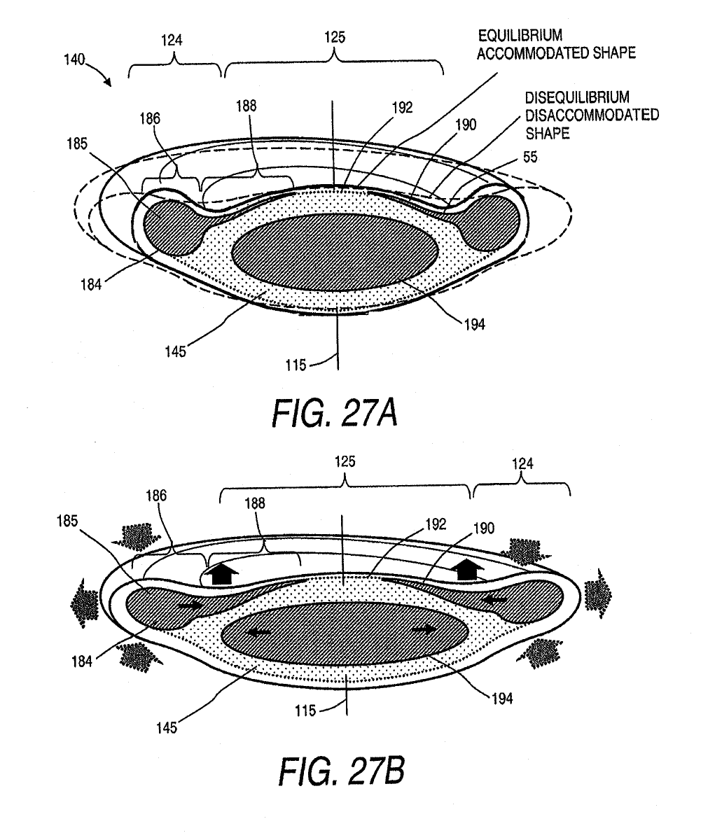

FIGS. 26A and 26B depict an alternative embodiment of lens 140 that again includes an interior block of a low modulus polymer 145 about optical axis 115. This embodiment again includes a symmetric interface 55 between elastomeric body portion 145 and an actuator feature, but in this case the actuator includes a displaceable media in the form of any index-matched fluid 184. In FIG. 26A, when the resilient polymer body is in its equilibrium or unstressed configuration, it can be seen that fluid or flowable media 184 is carried in a chamber 185 and more particularly a peripheral chamber portion 186 of the fluid-tight chamber 185. The use of a displaceable fluid 184 can allow for deformation of the surface curvature in the same manner as an ultralow modulus polymer, or allow for greater amplitude of deformation. As can be seen in FIG. 26B, the application of disaccommodating forces indicated by the block arrows (with dashed-line) about the non-optic portion 124 will cause inward displacement of flowable media 185 to increase the fluid volume in the inward chamber portion 188. In this embodiment, the chamber 185 comprise an annular interior region about axis 115 wherein the central portion of lens surface layer 190 is coupled to or bonded to interior elastomeric body portion 145 in constraining portion 192 which thus constrains the amplitude of deformation of the surface 190 about axis 115. At the same time, the more peripheral portion of the surface layer is unconstrained to allow it to substantially deform to flatten the lens and reduce the lens power. FIGS. 27A-27B illustrate another similar embodiment of lens 140 that differs only in that another interior chamber 195 is provided that carries a displaceable flowable media 185. In all other respects, the lens of FIGS. 27A-27B is similar to that of FIGS. 26A-26B.

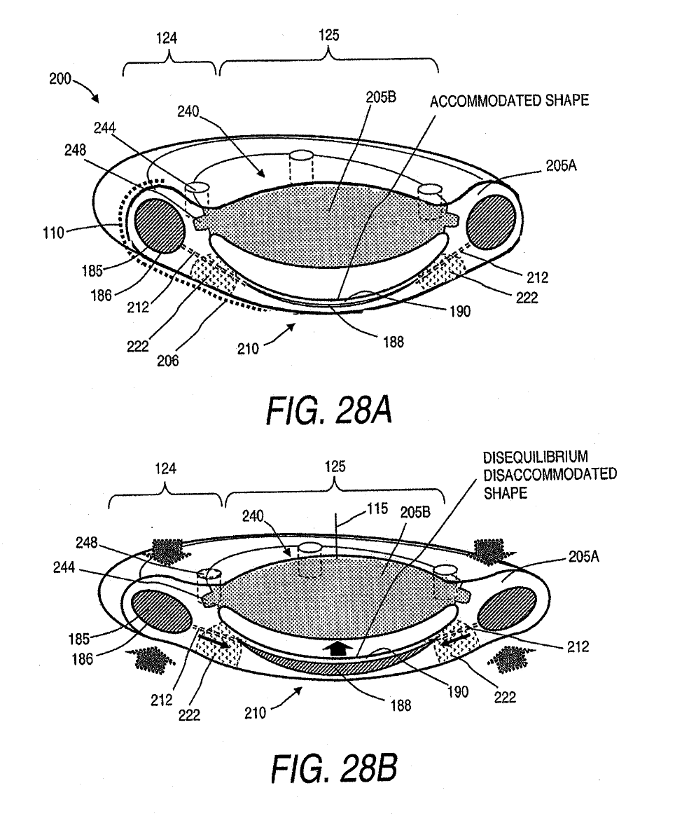

FIGS. 28A and 28B depict an alternative embodiment of lens 200 that is similar to that of FIGS. 15A-15C in that the lens implant has first and second components 205A and 205B that are interlocked after each component is injected independently into the capsular sac. The sequential introduction of first and second matable components thus allows for a smaller diameter injector. In one embodiment as in FIG. 28A, the first component 205A comprises an implant body as in FIG. 15C that has a peripheral portion 110 and posterior surfaces 206 for engaging the capsular sac shown with a dashed line. In the embodiment of FIG. 28A-28B, the first component 205A includes a deformable lens 210 that has a deformable anterior surface 190 that can be deformed by means of the displaceable fluid in peripheral chamber portion 186 that can be displaced to inward chamber portion 188, as in the embodiment of FIGS. 26A-26B. In this embodiment, the flow of fluid between the peripheral and inward chamber portions occurs within channels 212 wherein the peripheral portion of the lens is constrained by constraining structure or bond regions indicated at 222. The constraining structure 222 is depicted in the form of a bond between surface portions of polymeric substrates, but the scope of the invention extends to any form of constraining structure 222 which allows for a flow path within or proximate thereto, such as webs, tethers, foams, microfabricated materials, microchanneled materials and the like. Thus, the embodiment of FIGS. 28-28B provides a lens surface 190 with an unconstrained central portion and a constrained peripheral portion, which is the reverse of FIGS. 26A-26B. In the embodiment of FIGS. 28A-28B, the inward chamber portion about axis 115 is configured to alter the power of a negative power lens, which cooperates with the positive power lens 240 of second components 205B. The second component 205B is a fixed power drop-in lens than can be interlocked with first component 205A at interlock features 242, which can be of any type referred to above. In FIG. 28B, in can be seen that optic 210 and its deformable anterior surface 190 is actuated into space 244 between the lenses to flatten the negative power lens. At least one port 248 is provided around the interlock features of the lens components to allow aqueous flow into and about space 244. The functioning of embodiments such as in FIGS. 26A-26B and 28A-28B with a displaceable fluid 285 in an actuatable annular chamber portion or a central chamber portion to add or subtract power in either a positive or negative power lens was disclosed by the author in co-pending U.S. application Ser. No. 10/358,038, filed Feb. 3, 2003, which is incorporated herein.

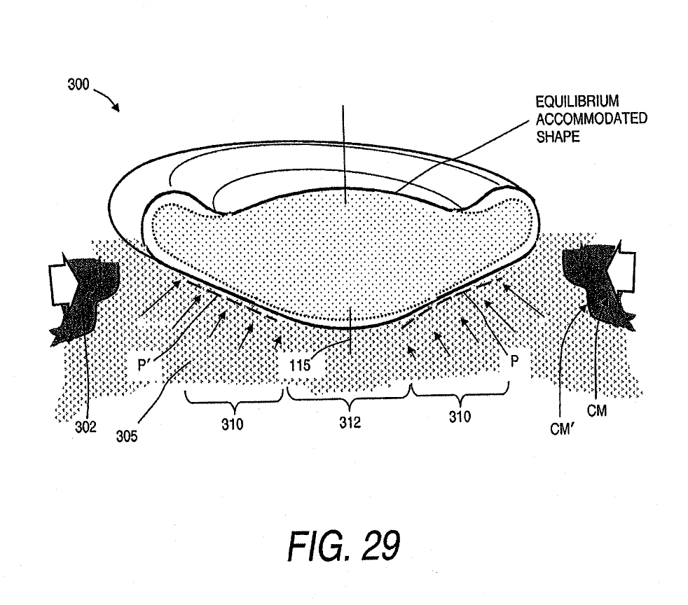

FIG. 29 depicts an alternative embodiment of lens 300 that is similar to the embodiments of FIGS. 20-28B which includes an implant surface shape that is configured to better respond to pressures caused by vitreous displacement that assist moving a natural lens capsule from a disaccommodated shape to an accommodated shape. Modeling suggests that impingement by ciliary muscles 302 on the gel-like vitreous body 305 (ciliary muscle surface movement from CM to CM′) causes anterior and inwardly directed forces that will vary over the posterior surface of the lens capsule, which forces also will differ over time as a patient's vitreous changes in its viscous properties. For this reason, as depicted in FIG. 29, the posterior surface of the lens 300 can have a flattened portion or plane 310 that extends annularly about axis 115 wherein plane 310 enhances the forces that are applied peripherally and inwardly to the lens 300 to assist in moving the lens toward its accommodated shape, and causes lesser anterior-directed forces to be applied to the central lens portion about axis 115. The vitreous can be considered an axisymmetric body wherein ciliary muscle impingement directs forces inwardly to the axis of the vitreous body wherein inward vitreous displacement exactly at the axis 115 is zero. Thus, anterior displacement of the vitreous will be greater at distances extending away from the axis 115. In one embodiment depicted in FIG. 29, the flattened plane 310 is substantially flat as indicated by line P in FIG. 29, or the flattened plane 310 can be concave as indicated by line P′ in FIG. 29, or can have a curvature that is substantially less than the central lens portion indicated at 312 in FIG. 29.

In another aspect, the business methods of fabricating and marketing accommodating intraocular lenses call with scope of the invention. One business method includes (i) fabricating an intraocular lens configured for implantation in a capsular sac, wherein the lens has interior displaceable media for adjusting a deformable lens surface between first and second powers, (ii) configuring a deformable lens surface for high amplitude axial deformation by the displaceable media about a periphery of a central optic portion and configuring the lens surface for low amplitude axial deformation about optical axis of the central optic portion, and (iii) collaboratively or independently, marketing the intraocular lens. In this business method, the fabricating step includes fabricating the displaceable media from a polymer having a modulus of less than 1000 KPa, less than 500 KPa and less than 100 KPa. An alternative business method includes fabricating the lens with a displaceable media that includes at least one interior chamber in the lens that carries flowable media.

In another aspect of the invention, a business method includes (i) fabricating an intraocular lens configured for implantation in a capsular sac, wherein the lens has a monolithic form with an exterior surface that continuously engages the interior of a capsular sac except the capsularhexis, (ii) configuring the lens with a central optic portion that is deformable between a first power and a second power in response to physiologic accommodating and disaccommodating forces applied to the lens capsule wherein the anterior surface of the optic portion is recessed within the monolithic form, and (iii) collaboratively or independently, marketing the intraocular lens. In another aspect of the invention, a business method includes (i) fabricating an intraocular lens configured for implantation in a capsular sac, wherein the lens has a monolithic form with an exterior surface that continuously engages the interior of a capsular sac except the capsularhexis, and wherein the form defines at least one interior on-axis, rotationally symmetric interface between at least two elastomeric blocks wherein each block has a different Young's modulus, and (ii) collaboratively or independently, marketing the intraocular lens.

In another aspect of the invention, a business method includes (i) fabricating an intraocular lens configured for implantation in a capsular sac, wherein the lens has a monolithic form with an exterior surface that continuously engages the interior of a capsular sac except the capsularhexis, and wherein the form defines a rotationally symmetric interface between at least two elastomeric blocks wherein one block is a shape memory polymer, and (ii) collaboratively or independently, marketing the intraocular lens.

The following commonly-owned U.S. Provisional Patent Applications are incorporated by reference herein and made a part of the specification: Appln. No. 60/547,408 filed Feb. 24, 2004, titled Ophthalmic Devices, Methods of Use and Methods of Fabrication; Appln. No. 60/487,541, filed Jul. 14, 2003, titled Ophthalmic Devices, Methods of Use and Methods of Fabrication; Appln. No. 60/484,888, filed Jul. 2, 2003, titled Ophthalmic Devices, Methods of Use and Methods of Fabrication; Appln. No. 60/480,969, filed Jun. 23, 2003, titled Ophthalmic Devices, Methods of Use and Methods of Fabrication; Appln. No. 60/353,847, filed Feb. 6, 2002, titled Intraocular Lens and Method of Making; Appln. No. 60/362,303, filed Mar. 6, 2002, titled Intraocular Lens and Method of Making; Appln. No. 60/378,600, filed May 7, 2002, titled Intraocular Devices and Methods of Making; Appln. No. 60/405,471, filed Aug. 23, 2002, titled Intraocular Implant Devices and Methods of Making, Appln. No. 60/408,019, filed Sep. 3, 2002, titled Intraocular Lens, and Appln. No. 60/431,110, filed Dec. 4, 2002, titled Intraocular Implant Devices and Methods of Making.

Those skilled in the art will appreciate that the exemplary systems, combinations and descriptions are merely illustrative of the invention as a whole, and that variations in the dimensions and compositions of invention fall within the spirit and scope of the invention. Specific characteristics and features of the invention and its method are described in relation to some figures and not in others, and this is for convenience only. While the principles of the invention have been made clear in the exemplary descriptions and combinations, it will be obvious to those skilled in the art that modifications may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

What is claimed is:

1. An intraocular lens configured for implantation in a capsular sac, the lens capable of accommodative deformation between first and second dioptic strengths in response to shape changes of the capsular sac, the lens comprising:
a first material disposed symmetrically about an optical axis, the first material having a first elastic modulus; and
a second material structure having an annular configuration outward of the first material, the second material structure having a second elastic modulus which is higher than the first elastic modulus;
wherein shape changes in the capsular sac cause relative displacement of the first and second materials to thereby alter the lens between the first and second dioptric strengths.

2. The intraocular lens of claim 1 wherein the relative displacement is radial relative to the optical axis.

3. The intraocular lens of claim 1 wherein the relative displacement is axial relative to the optical axis.

4. The intraocular lens of claim 1 wherein the relative displacement is both radial and axial relative to the optical axis.

5. The intraocular lens of claim 1 wherein the first and second material have substantially matching indices of refraction.

6. The intraocular lens of claim 1 wherein the first elastic modulus is between zero KPa and about 200 KPa.

7. The intraocular lens of claim 1 wherein the second material is disposed at least partly about a surface of the lens body.

8. The intraocular lens of claim 1 wherein the second material structure extends across the posterior of the first material.

9. The intraocular lens of claim 1 wherein the cross section of the second material tapers toward the optical axis.

10. The intraocular lens of claim 1 wherein the second material includes a plurality of radially spaced segments.

11. The intraocular lens of claim 1 wherein the second material at least partly extends within an interior of the lens.

12. The intraocular lens of claim 1 wherein the second material at least partly extends within an interior of the first material.

13. The intraocular lens of claim 1 wherein said second elastic modulus is between about 50 KPa and 400 KPa.

14. The intraocular lens of claim 1 wherein the first material comprises a fluid.

15. An intraocular lens (IOL) configured for implantation in a lens capsule, the IOL capable of accommodative deformation between first and second dioptric strengths in response to capsular shape changes, the IOL comprising:
a lens body with a first portion centered about an optical axis; and
a second portion having an annular configuration symmetrically positioned relative to the centered first portion, the second portion configured for transducing forces from capsular shape changes from the second portion to the first portion;
wherein the transduced forces include forces applied by at least one of radial and axial movements of the second portion.

16. The intraocular lens of claim 15 wherein the first and second portions have substantially different elastic moduli.

17. The intraocular lens of claim 15 wherein the first portion has a lower elastic modulus than the second portion.

18. The intraocular lens of claim 15 wherein the first and second portions have substantially matching indices of refraction.

19. The intraocular lens of claim 15 wherein said first portion elastic modulus is between zero and about 200 KPa.

20. The intraocular lens of claim 15 wherein said second portion elastic modulus is between about 50 KPa and 400 KPa.

21. The intraocular lens of claim 15 wherein the second portion is disposed at least partly about a surface of the lens body.

22. The intraocular lens of claim 15 wherein the second portion extends across the posterior of the first material.

23. The intraocular lens of claim 15 wherein the second portion is disposed at least partly in an interior of the lens body.

24. The intraocular lens of claim 15 wherein said first and second portions include a bonded interface therebetween.

25. The intraocular lens of claim 15 wherein said first and second portions are matable in situ.

26. The intraocular lens of claim 15 wherein the first portion comprises a fluid.

27. An intraocular lens (IOL) configured for implantation in a lens capsule, the IOL capable of accommodative deformation between a lens-accommodated state and a lens-disaccommodated state in response to capsular shape changes, the IOL comprising:
a deformable lens body having a central body portion and a peripheral body structure coupled about an annular interface outward of an optical axis, the lens body in equilibrium in the lens-accommodated state; and
wherein capsular shape changes displace the central body portion about the optical axis relative to the peripheral body structure to move the lens body to the lens-disaccommodated state.

28. The intraocular lens of claim 27 wherein the central body portion comprises a liquid.

29. The intraocular lens of claim 27 wherein the central body portion comprises a polymer.

30. The intraocular lens of claim 27 wherein the peripheral body structure radially decreases in thickness toward the annular interface.

31. The intraocular lens of claim 27 wherein the peripheral body structure radially increases in flexibility toward the annular interface.

32. An intraocular lens (IOL) configured for implantation in a lens capsule, the IOL capable of accommodative deformation between a lens-accommodated state and a lens-disaccommodated state in response to capsular shape changes, the IOL comprising:
a deformable lens body having a central body portion and a peripheral body structure coupled about an annular interface outward of an optical axis; and
wherein capsular shape changes (i) displace the central body structure relative to the optical axis a first amount and (ii) displace the peripheral body structure relative to the optical axis a second greater amount to move the lens body between the lens-accommodated and lens-disaccommodated states.
